(19) United States
(12) Patent Application Publication
(10) Pub. No.: US 2025/0090771 A1
(43) Pub. Date: Mar. 20, 2025

(54) ELECTRONIC DEVICES AND LIQUIDS FOR AEROSOLIZING AND INHALING THEREWITH

(71) Applicant: QNOVIA, INC., RICHMOND, VA (US)

(72) Inventors: Mario Danek, Austin, TX (US); Ian Kovacevich, Carlsbad, CA (US); Andrew Heinrich, Denair, CA (US); Christopher Kar-Heng Cheng, Los Angeles, CA (US); Joseph Walsh, Los Angeles, CA (US)

(21) Appl. No.: 18/964,602

(22) Filed: Dec. 1, 2024

Related U.S. Application Data
(63) Continuation of application No. 17/075,679, filed on Oct. 20, 2020.
(60) Provisional application No. 62/923,563, filed on Oct. 20, 2019.
(60) Provisional application No. 62/923,602, filed on Oct. 20, 2019.
(60) Provisional application No. 62/923,604, filed on Oct. 20, 2019.
(60) Provisional application No. 62/924,168, filed on Oct. 21, 2019.
(60) Provisional application No. 62/924,171, filed on Oct. 21, 2019.

(51) Int. Cl. A61M 11/00; A24B 15/167; A24F 40/05; A24F 40/10; A24F 40/42; A61K 9/00; A61K 9/107; A61K 9/127; A61K 31/00; A61K 31/05; A61K 31/352; A61K 31/4439; A61K 31/465; A61K 47/02; A61K 47/26; B05B 17/06

(52) U.S. Cl. CPC A61M 11/003; A24B 15/167; A24F 40/05; A24F 40/10; A24F 40/42; A61K 9/0078; A61K 9/1075; A61K 9/127; A61K 31/05; A61K 31/352; A61K 31/4439; A61K 31/465; A61K 31/658; A61K 47/02; A61K 47/26; B05B 17/0653; A61M 2205/0294; A61M 2205/12; A61M 2205/587; A61M 2205/8206

(57) ABSTRACT

An electronic device includes a mouthpiece, a bladder, and a mesh assembly having a mesh material and a piezoelectric material. The mesh material is in contact with a liquid of the bladder. The mouthpiece, the bladder, and the mesh assembly are located in-line along a longitudinal axis of the device between opposite longitudinal ends of the device, with the mesh assembly extending between and separating the mouthpiece and the bladder. A liquid-filled cartridge also is disclosed for use with an electronic device for delivery of a substance into a body through respiration includes a liquid container; and a liquid contained within the container for aerosolizing and inhaling by a person using the electronic device. The liquid includes a plurality of nanoparticles in a nanoemulsion, the nanoparticles including the encapsulation of the substance to be delivered into the body through respiration. The nanoemulsion preferably is produced using a microfluidizing machine.

### CROSS-REFERENCE TO APPLICATIONS

This application claims incorporates by reference each of U.S. provisional patent applications: 62/923,563, filed Oct. 20, 2019; 62/923,602, filed Oct. 20, 2019; 62/923,604, filed Oct. 20, 2019; 62/924,168, filed Oct. 21, 2019; and 62/924,171, filed Oct. 21, 2019, each of which is incorporated herein by reference. This application also incorporates by reference Applicant's U.S. patent applications Ser. Nos. 16/548,831; 16/657,732; and 16/657,755, and any U.S. patent application publication thereof and any U.S. patent issuing therefrom. Aspects and features of the invention are believed to be improvements and enhancements over the devices and methods of Applicant's '831, '732, and '755 applications.

### COPYRIGHT STATEMENT

All of the material in this patent document is subject to copyright protection under the copyright laws of the United States and other countries. The copyright owner has no objection to the facsimile reproduction by anyone of the patent document or the patent disclosure, as it appears in official governmental records but, otherwise, all other copyright rights whatsoever are reserved.

### COMPUTER PROGRAM LISTING

Submitted concurrently herewith via the USPTO's electronic filing system, and incorporated herein by reference, are computer program files. A table setting forth the name and size of files included in this computer program listing is included below.

| File Name | Creation Date | File Size (bytes) |
|---|---|---|
| ascify.txt | Oct. 20, 2019 20:33 | 37,473 |
| readme.txt | Oct. 20, 2019 11:55 | 2,741 |
| files1.txt | Oct. 20, 2019 20:41 | 22,478,505 |
| files2.txt | Oct. 20, 2019 20:59 | 11,960,834 |

One of these files, “readme.txt”, contains instructions for extracting information from “files1.txt” and “files2.txt”. “files1.txt” and “files2.txt” collectively represent a compressed binary file that has been converted to ascii format. These files can be converted back to a compressed .zip archive utilizing an assembly conversion program source code for which is contained in “ascify.txt”. The readme file includes instructions for compiling and running this conversion program, and instructions for converting the other text files to a compressed, binary file.

This compressed, binary file includes eDrawings files for a computer model illustrating aspects and features in accordance with one or more preferred embodiments, as well as a .pdf file illustrating aspects and features in accordance with one or more preferred embodiments.

### BACKGROUND OF THE INVENTION

The invention generally relates to apparatus, systems, and methods for producing an aerosol for inhalation by a person, whether intended for personal or recreational use, or for the administration of medicines.

Vaping has been rapidly increasing in popularity, primarily because vaping provides a convenient, discreet, and presumably benign way to self-administer nicotine, cannabis, drugs or other micronutrients. Indeed, there is a common belief that vaping is healthier than smoking cigarettes; vaping purportedly lets smokers avoid dangerous chemicals inhaled from regular cigarettes while still getting nicotine. Vaping also can be used for cannabis.

Vaping is performed using a vaporizer. A vaporizer includes a vape pen or a cigarette style vape, referred to by many as an e-cigarette or “eCig”. A vape pen generally is an elongate, thin, and stylized tube that resembles a fancy pen. In contrast, an e-cigarette resembles an actual cigarette. The e-cigarette is usually small in size (usually smaller and more discreet than vape pens), easily portable, and easy to use.

A common vaporizer comprises a container, which may be a tank—which is typically refillable, or a cartridge—which is typically single-use and not refillable. The tank or cartridge holds a liquid often referred to as an e-liquid or e-juice. Tanks are made out of polycarbonate plastic, glass, or stainless steel. The vaporizer also includes a mouthpiece for inhaling by a person through the mouth; an atomizer comprising a tiny heating element that converts the liquid into tiny, airborne droplets that are inhaled; and a controller for turning on the atomizer. Many vape pens are mouth-activated and turn on automatically when a person inhales. Others vape pins are button activated and require the person to push a button to activate the atomizer. Vaporizers are electrically powered using one or more batteries. The batteries typically are lithium ion batteries that are rechargeable and primarily are used to heat the heating element of the atomizer. A charger usually accompanies a vaporizer when purchased for charging the batteries. The charger may be a USB charger, car charger, or wall charger, and such chargers are generally similar to phone chargers.

The battery-powered vaporizer produces vapor from any of a variety of liquids and liquid mixtures, especially those containing nicotine or cannabinoids. Many different types and flavors are available. Moreover, the liquids can be non-medicated (i.e., containing no nicotine or other substances—just pure vegetable glycerin and flavoring), or the liquids can contain nicotine or even in some instances if and where legal, the liquids can contain THC/CBD. The liquids also may contain one or more of a variety of flavors as well as micronutrients such as, for example, vitamin B12. A person can mix the liquids for use with a vape pen. E-cigarettes typically are purchased with prefilled cartridges. The heating element turns the contents of the liquids into an aerosol—the vapor—that is inhaled into the lungs and then exhaled by the person. Perhaps one of the most popular vaporizers today is known as the “JUUL”, which is a small, sleek device that resembles a computer USB flash drive.

It is believed that while promoted as healthier than traditional cigarette use, vaping actually may be more dangerous. Propylene glycol, vegetable glycerin and combinations or methylations thereof, are chemicals that are often mixed with nicotine, cannabis, or hemp oil for use in vaporizers. Propylene glycol is the primary ingredient in a majority of nicotine-infused e-cigarette liquids. Unfortunately, at high temperatures propylene glycol converts into tiny polymers that can wreak havoc on lung tissue. In particular, scientists know a great deal about propylene glycol. It is found in a plethora of common household items—cosmetics, baby wipes, pharmaceuticals, pet food, antifreeze, etc. The U.S. Food and Drug Administration and Health Canada have deemed propylene glycol safe for human ingestion and topical application. But exposure by inhalation is another matter. Many things are safe to eat but dangerous to breathe. Because of low oral toxicity, propylene glycol is classified by the FDA as “generally recognized as safe” (GRAS) for use as a food additive, but this assessment was based on toxicity studies that did not involve heating and breathing propylene glycol. Indeed, a 2010 study published in the International Journal of Environmental Research and Public Health concluded that airborne propylene glycol circulating indoors can induce or exacerbate asthma, eczema, and many allergic symptoms. Children were said to be particularly sensitive to these airborne toxins. An earlier toxicology review warned that propylene glycol, ubiquitous in hairsprays, could be harmful because aerosol particles lodge deep in the lungs and are not respirable.

Moreover, when propylene glycol is heated, whether by a red-hot metal coil of a heating element of a vaporizer or otherwise, the potential harm from inhalation exposure increases. It is believed that high voltage heat transforms the propylene glycol and other vaping additives into carbonyls. Carbonyls are a group of cancer-causing chemicals that includes formaldehyde, which has been linked to spontaneous abortions and low birth weight. A known thermal breakdown product of propylene glycol, formaldehyde is an International Agency for Research on Cancer group 1 carcinogen!

Prevalent in nicotine e-cig products and present in some vape oil cartridges, FDA-approved flavoring agents pose additional risks when inhaled rather than eaten. The flavoring compounds smooth and creamy (diacetyl and acetyl propionyl) are associated with respiratory illness when inhaled in tobacco e-cigarette devices. Another hazardous-when-inhaled-but-safe-to-eat flavoring compound is Ceylon cinnamon, which becomes cytotoxic when aerosolized.

When a heating element gets red hot in a vaporizer, the liquid undergoes a process called “smoldering”, which is a technical term for what is tantamount to “burning”; while much of the liquid is vaporized and atomized, a portion of the liquid undergoes pyrolysis or combustion. In that sense, most of the vaporizers that have flooded the commercial market may not be true vaporizers.

Additionally, clearance mechanisms of the lung, like all major points of contact with the external environment, have evolved to prevent the invasion of unwanted airborne particles from entering the body. Airway geometry, humidity and clearance mechanisms contribute to this filtration process.

In view of the foregoing, it is believed that a need exists for a vaporizer that provides an aerosol of the desired chemicals without the harmful byproducts that arise from smoldering. It is also believed that a need exists for a vaporizer that effectively and efficiently produces a vapor cloud that is not inhibited by the body's natural filtration process. This and other needs are believed to be met by embodiments in accordance with one or more aspects and features of the invention.

Furthermore, the invention also generally relates to apparatus, systems, formulations, and methods pertaining to liquids that are aerosolized and inhaled by persons using electronic devices, whether intended for personal or recreational use, or for the administration of medicines.

Inhalation delivery systems now play an increasing role in the targeted delivery of active ingredients to the human pulmonary system. This is true both for medical purposes, such as the targeted delivery of anti-cancer medications to the lungs, as well as for recreational/personal purposes, such as vaping, in which a liquid that includes the active ingredient is vaporized using heating so that the active ingredient can be inhaled into the human body.

Unfortunately, as inhalation delivery systems using heating have increased in prominence, concerns about their short and long term safety have come into focus. This is particularly true for vaping where there exist ongoing concerns about the possible presence of harmful and potentially harmful constituents (HPHCs) in the inhaled vapor. Moreover, inhalation delivery systems are often unable to provide the desired effect to a user. This may be attributable to the pre-vaporized liquid becoming unstable over time or the active ingredient itself not being properly sized or dispersed for deposition in the alveolar lung.

Accordingly, a need exists for an active ingredient delivery system that enhances the shelf-life of the pre-vaporized liquid component and enhances the efficacy of the desired treatment/effect, while avoiding the presence of undesired HPHCs in the inhaled vapor. This, and other needs, are believed to also be addressed by one or more aspects and features of the invention.

### SUMMARY OF THE INVENTION

The invention includes many aspects and features. Moreover, while many aspects and features relate to, and are described in, the context of vaping, the invention is not limited to use only in such context. Indeed, depending on the context of use, the electronic device of the invention may be considered a vaporizer and may be in the form of a vape pen or e-cigarette. Indeed, those who vape may come to refer to embodiments of the invention as a vape pen even though heat is not utilized to create the aerosol that is inhaled. In the delivery of pharmaceuticals, patients may come to refer to embodiments of the invention as a nebulizer even though a gas transport (e.g., compressed gas) is not utilized and even though the aerosol that is produced in accordance with the invention may have a smaller particle size than the mist produced by common nebulizers. Other separate and distinct contexts of use of embodiments of the invention may similarly result in different nomenclature of the embodiments of the invention. Nonetheless, while the appearance and form factor of embodiments of the invention may vary depending on such contexts of use, the basic components and operation remain the same, except where otherwise described below.

Some embodiments in accordance with aspects and features of the present invention preferably utilize a bladder for supplying the liquid to the mesh assembly, the piezoelectric material of which aerosolizes the liquid for inhalation by a person. The bladder may be used in replacement of the “liquid container” of Applicant's '831, '732, and '755 applications, with the bladder preferably forming a part of a disposable cartridge. Additionally, the bladder preferably is formed from a self-healing material such as silicone and is filled with the fluid by injection. The injection process preferably occurs during the manufacture of the cartridge after the bladder has been formed and installed into the cartridge. The injection site of the bladder preferably is the end of the bladder distally located to the port of the mouthpiece through which the aerosol is inhaled. Alternatively, the injection site of the bladder may be located to a side of the bladder. Various shapes and sizes of bladders are disclosed in the current application, including collectively the drawings and the eDrawings and PDF files of the computer program listing, which is incorporated herein by reference and which forms part of the disclosure of the present application.

Aspects of the invention also comprises using an electronic device of the present invention to produce an aerosol for inhalation by a person using such electronic device.

Additional features of the invention are set forth in any and each incorporated application of Applicant, including any incorporated U.S. patent application publication thereof and any incorporated U.S. patent issuing therefrom.

In another aspect, a liquid-filled cartridge for use with an electronic device for delivery of a substance into a body through respiration comprises: a liquid container; and (b) a liquid contained within the container for aerosolizing and inhaling by a person using the electronic device, the liquid comprising a plurality of nanoparticles in a nanoemulsion, each nanoparticle comprising an encapsulation of the substance to be delivered into the body through respiration.

In a feature, the liquid is an oil-in-water nanoemulsion.

In a feature, each nanoparticle is a micelle.

In a feature, each nanoparticle is a liposome.

In a feature, the substance is encapsulated by a polymer.

In a feature, the substance is encapsulated by a surfactant. The surfactant preferably comprises high purity polyoxyethylene sorbitan monooleate.

In a feature, the encapsulated substance comprises tetrahydrocannabinol.

In a feature, the encapsulated substance comprises cannabidiol.

In a feature, the encapsulated substance comprises tetrahydrocannabinol and cannabidiol.

In a feature, the encapsulated substance comprises a pharmaceutical compound.

In a feature, the encapsulated substance comprises nicotine.

In a feature, wherein the nanoparticles are suspended within an aqueous solution. The aqueous solution preferably comprises a saline; the aqueous solution preferably comprises sodium chloride; and, the nanoparticles preferably are suspended within an aqueous solution of 0.9% sodium chloride.

In a feature, a pH of the liquid is between about 5.5 and about 8.

In a feature, wherein a pH of the liquid is between about 6.5.

In a feature, a molecular ratio of the encapsulated substance to an encapsulating agent of the nanoparticle between about 0.1:1 to about 10:1.

In a feature, a polydispersity index measurement of the liquid is less than 0.3.

In a feature, the cartridge is a single-use, disposable cartridge.

In a feature, the cartridge is refillable.

In another aspect, a method of manufacturing cartridges for use with an electronic device for delivery of a substance into a body through respiration comprises filling a liquid container of the cartridge with a liquid for aerosolizing and inhaling by a person using the electronic device, the liquid comprising a plurality of nanoparticles in a nanoemulsion, each nanoparticle comprising an encapsulation of the substance to be delivered into the body through respiration.

In a feature, the method further comprises a preliminary step of producing the nanoemulsion by processing the substance to be delivered together with the encapsulating agent using a microfluidizing machine.

In a feature, the method further comprises operating the microfluidizing machine such that a temperature of the processing does not exceed 65° C. while producing the nanoemulsion.

In a feature, the method further comprises the step of adjusting pH of the nanoemulsion so as to be between about 5.5 and 8.

In a feature, the method further comprises the step of chemically bonding the substance to be encapsulated with another molecule prior to processing the substance with the encapsulating agent using the microfluidizing machine. The polydispersity index measurement of the nanoemulsion after processing using the microfluidizing machine preferably is less than 0.3.

In another aspect, a method of manufacturing a liquid for aerosolizing and inhaling by a person using an electronic device for the delivery of a substance to the body of the person through respiration, the method comprising producing a liquid comprising a plurality of nanoparticles in a nanoemulsion by processing the substance together with an encapsulating agent using a microfluidizing machine such that the plurality of nanoparticles of the liquid comprises the encapsulated substance.

In a feature, the method further comprises operating the microfluidizing machine such that a temperature of the processing does not exceed 65° C. while producing the liquid.

In a feature, the method further comprises adjusting pH of the nanoemulsion so as to be between about 5.5 and 8.

In a feature, the method further comprises the step of chemically bonding the substance to be encapsulated with another molecule prior to processing the substance with the encapsulating agent using the microfluidizing machine.

In a feature, a polydispersity index measurement of the nanoemulsion after processing using the microfluidizing machine is less than 0.3.

Another aspect of the invention relates to a liquid formulation for aerosolization. The liquid formulation includes an aqueous solution, one or more encapsulating agents, and an active ingredient.

In a feature of this aspect, the active ingredient is encapsulated by one or more encapsulating agents to form a nanoparticle. In another feature of this aspect, the nanocarrier comprises a liposome. In still another feature of this aspect, the nanocarrier comprises a micelle.

In another feature of this aspect, the nanoparticles have an average diameter of less than 1,000 nanometers.

In another feature of this aspect, the one or more encapsulating agents comprise a polymer. In another feature of this aspect, the one or more encapsulating agents comprise a surfactant. In still another feature of this aspect, the surfactant comprises a high purity polyoxyethylene sorbitan monooleate, such as “SUPER REFINED Polysorbate 80”.

In another feature of this aspect, the aqueous solution comprises a saline solution. In another feature of this aspect, the saline solution comprises a 0.9% saline solution.

In another feature of this aspect, the active ingredient comprises tetrahydrocannabinol. In another feature of this aspect, the active ingredient comprises cannabidiol. In another feature of this aspect, the active ingredient comprises tetrahydrocannabinol and cannabidiol. In another feature of this aspect, the active ingredient comprises nicotine. In still another feature of this aspect, the active ingredient comprises a pharmaceutical compound.

In another feature of this aspect, a ratio of the one or more encapsulating agents to the active ingredient is between about 0.1:1 to about 10:1.

In another feature of this aspect, a pH measurement of the liquid formulation is between about 5.5 and about 8. In another feature of this aspect, a pH measurement of the liquid formulation is about 6.5.

In another feature of this aspect, a polydispersity index measurement of the liquid formulation is less than 0.3.

In another feature of this aspect, the active ingredient is chemically bonded to another molecule.

Another aspect of the invention relates to a method of preparing a liquid formulation for aerosolization. The method comprises the steps of mixing nanoparticles that include an active ingredient in a solution to form a liquid mixture and processing the liquid mixture with a microfluidizer.

In a feature of this aspect, a temperature of the liquid mixture does not exceed 65° C. during the processing step.

In another feature of this aspect, the method further comprises the step of adjusting the pH of the liquid mixture.

In another feature of this aspect, the method further comprises the step of chemically bonding the active ingredient with another molecule.

In another feature of this aspect, nanoparticles of the microfluidized liquid mixture have an average diameter less than 1,000 nanometers.

In another feature of this aspect, a polydispersity index measurement of the microfluidized liquid mixture is less than 0.3.

In another feature of this aspect, the solution comprises an aqueous solution. In another feature of this aspect, the aqueous solution comprises a 0.9% saline solution.

In another feature of this aspect, the nanoparticles comprise encapsulated nanoparticles. In another feature of this aspect, the active ingredient is contained within the encapsulated nanoparticles. In another feature of this aspect, the nanocarrier comprises a liposome. In still another feature of this aspect, the nanocarrier comprises a micelle.

In another feature of this aspect, the active ingredient comprises tetrahydrocannabinol. In another feature of this aspect, the active ingredient comprises cannabidiol. In another feature of this aspect, the active ingredient comprises tetrahydrocannabinol and cannabidiol. In another feature of this aspect, the active ingredient comprises nicotine. In still another feature of this aspect, the active ingredient comprises a pharmaceutical compound.

In addition to the aforementioned aspects and features of the invention, it should be noted that the invention further encompasses the various logical combinations and subcombinations of such aspects and features. Thus, for example, claims in this or a divisional or continuing patent application or applications may be separately directed to any aspect, feature, or embodiment disclosed herein, or combination thereof, without requiring any other aspect, feature, or embodiment.

### BRIEF DESCRIPTION OF THE DRAWINGS

One or more preferred embodiments of the invention now will be described in detail with reference to the accompanying drawings, wherein the same elements are referred to with the same reference numerals.

FIG. 1 is a perspective view of a preferred embodiment of a vaporizer in accordance with one or more aspect and features of the invention.

FIG. 2 is a partial view of the vaporizer of FIG. 1 showing in closeup a counter, battery indicator, and mouthpiece thereof.

FIG. 3 is another perspective view of the vaporizer of FIG. 1.

FIG. 4 is still yet another perspective view of the vaporizer of FIG. 1.

FIG. 5 is a perspective view of the other side of the vaporizer seen in FIG. 1.

FIG. 6 is a perspective view of one of two opposite ends of the vaporizer of FIG. 1, which illustrated end comprises the mouthpiece of the vaporizer.

FIG. 7 is an exploded view of the vaporizer of FIG. 1. The bladder can be seen illustrated in blue with the piezo mesh disk and electrical contacts attached to form the mesh assembly. The mesh assembly is retained within a body of the cartridge, which cartridge is seen to have a perforated band in FIG. 7.

FIG. 8 is a vapor cloud that is produced by a push of the button of the vaporizer of FIG. 1, which vapor cloud preferably has a known quantity of the substance to be inhaled per push of the button/aerosolizing cycle of the vaporizer.

FIG. 9a is a solid, perspective view of an end of a second preferred embodiment of a vaporizer in accordance with one or more aspects and features of the invention.

FIG. 9b is another solid, perspective view of the vaporizer of FIG. 9a.

FIG. 9c is a solid, perspective view of an end of the vaporizer opposite to the end shown in FIGS. 9a and 9b.

FIG. 10a is a solid line drawing of the view seen in FIG. 9a.

FIG. 10b is a solid line drawing of the view seen in FIG. 9b.

FIG. 10c is a solid line drawing of the view seen in FIG. 9c.

FIG. 11a is a line drawing of the view seen in FIG. 9a.

FIG. 11b is a line drawing of the view seen in FIG. 9b.

FIG. 11c is a line drawing of the view seen in FIG. 9c.

FIG. 12a is a solid, perspective view of the opposite end of the second preferred embodiment, which end is the subject of focus in FIG. 9c.

FIG. 12b is another solid, perspective view of the end of the vaporizer of FIG. 12a.

FIG. 12c is another solid, perspective view of the end of the vaporizer of FIG. 12a.

FIG. 13a is a solid line drawing of the view seen in FIG. 12a.

FIG. 13b is a solid line drawing of the view seen in FIG. 12b.

FIG. 13c is a solid line drawing of the view seen in FIG. 12c.

FIG. 14a is a line drawing of the view seen in FIG. 12a.

FIG. 14b is a line drawing of the view seen in FIG. 12b.

FIG. 14c is a line drawing of the view seen in FIG. 12c.

FIG. 15a is a solid, perspective view of a side of the vaporizer of FIG. 12a, which side includes the button.

FIG. 15b is a solid, plan view of a top end of the vaporizer of FIG. 12a.

FIG. 15c is a solid, plan view of the bottom end of the vaporizer of FIG. 12a.

FIG. 16a is a solid line drawing of the view seen in FIG. 15a.

FIG. 16b is a solid line drawing of the view seen in FIG. 15b.

FIG. 16c is a solid line drawing of the view seen in FIG. 15c.

FIG. 17a is a line drawing of the view seen in FIG. 15a.

FIG. 17b is a line drawing of the view seen in FIG. 15b.

FIG. 17c is a line drawing of the view seen in FIG. 15c.

FIG. 18a is a solid perspective view of the vaporizer of FIG. 12a.

FIG. 18b is another solid side view of the vaporizer of FIG. 12a.

FIG. 19a is a solid line drawing of the view seen in FIG. 18a.

FIG. 19b is a solid line drawing of the view seen in FIG. 18b.

FIG. 20a is a line drawing of the view seen in FIG. 18a.

FIG. 20b is a line drawing of the view seen in FIG. 18b.

FIGS. 21a, 21b, and 21c illustrate filling of a bladder of the cartridge after the bladder has been installed in the cartridge by injecting fluid directly into the bladder using a needle.

FIGS. 21d, 21e, 21f, 21g, 21h, and 21i illustrate mounting of the cartridge to a base of the vaporizer of FIGS. 1-8.

FIG. 22 is a perspective view of a preferred embodiment of a self-healing, silicone bladder after injection molding thereof in accordance with one or more aspects and features of the invention. The bladder of FIG. 22 has a capacity of about 2.5 milliliters. In other embodiments, the volume of the bladder is as much as 0.35 milliliters.

FIG. 23 is a partial perspective view of an end of a preferred embodiment of a vaporizer in accordance with one or more aspects and features of the invention, which end comprises a mouthpiece of the vaporizer.

FIG. 24a is a view of the vaporizer as seen in FIG. 23 wherein the mouthpiece has been removed to reveal a piezo mesh disk of FIG. 23. As seen in FIG. 24a, the piezo mesh disk is received with a cartridge body.

FIG. 24b is a transparent view of the vaporizer as seen in FIG. 24a, which reveals a bladder and the mesh assembly including the piezo mesh disk contained within a cartridge body in accordance with one or more aspects and features of the invention.

FIG. 25 is a perspective front view of the end of the vaporizer as seen in FIG. 24a, wherein the cartridge body and a main body casing have been removed to reveal the bladder secured to a mounting plate of the cartridge that, in turn, is secured to a main body chassis of the vaporizer.

FIG. 26 is another view of the vaporizer as seen in FIG. 24a, wherein the piezo mesh disk has been removed to reveal a mouth of the bladder.

FIG. 27a is another view of the vaporizer as seen in FIG. 26, wherein just the cartridge body and bladder are shown.

FIG. 27b is a bottom plan view of the cartridge as seen in FIG. 27a.

FIG. 27c is a perspective view of the bladder of the cartridge of FIG. 27a, which bladder is seen secured to the cartridge mounting plate.

FIG. 27d is a perspective view of just the cartridge mounting plate as seen in FIG. 27c.

FIG. 28a is a perspective back view of the vaporizer as seen in FIG. 25.

FIG. 28b is an elevational front view of the vaporizer as seen in FIG. 28a.

FIG. 28c is an elevational first side view of the vaporizer as seen in FIG. 28a.

FIG. 28d is an elevational back view of the vaporizer as seen in FIG. 28a.\

FIG. 28e is an elevational second side view of the vaporizer as seen in FIG. 28a.

FIG. 29a is a bottom perspective view of the bladder and mesh assembly, the cartridge

mounting plate, and magnets of the cartridge by which the mounting plate is secured to the main body chassis.

FIG. 29b is a top perspective view of the bladder and mesh assembly, the cartridge mounting plate, and magnets of the cartridge seen in FIG. 29a.

FIG. 29c is a back perspective view of the bladder and the mesh assembly, the cartridge mounting plate, and magnets of the cartridge of FIG. 29a.

FIG. 29d is a perspective elevational view of the bladder and the mesh assembly, the cartridge mounting plate, and magnets of the cartridge of FIG. 29a.

FIG. 29e is another back perspective view of the bladder and the mesh assembly, the cartridge mounting plate, and magnets of the cartridge of FIG. 29a.

FIG. 29f is a back elevational view of the bladder and the mesh assembly, the cartridge mounting plate, and magnets of the cartridge of FIG. 29a.

FIG. 30a is a front perspective view of the bladder and the mesh assembly of FIG. 29a without the cartridge mounting plate and magnets.

FIG. 30b is a bottom perspective view of the bladder and the mesh assembly of FIG. 30a.

FIG. 30c is a back perspective view of the bladder and the mesh assembly of FIG. 30a.

FIG. 30d is a back perspective view of the mesh assembly of FIG. 30a without the bladder.

FIG. 30e is a back perspective view of the bladder of FIG. 30a without the mesh assembly.

FIG. 30f is a bottom plan view of the bladder of FIG. 30e.

FIG. 30g is a side elevational view of the bladder of FIG. 30e.

FIG. 30h is a bottom perspective view of the bladder of FIG. 30e.

FIG. 30i is a top plan view of the bladder of FIG. 30a.

FIG. 31a is a bottom perspective view of an alternative bladder secured to the cartridge mounting plate of FIG. 29a.

FIG. 31b is an exploded view of the alternative bladder and mounting plate of FIG. 31a.

FIG. 31c is yet another alternative bladder secured to the cartridge mounting plate of FIG. 29a, which view is a shaded line drawing.

FIG. 31d is a solid view of the view of FIG. 31c.

FIG. 32a is a top perspective view of another alternative bladder for use with the cartridge mounting plate of FIG. 29a.

FIG. 32b is a bottom perspective view of the bladder of FIG. 32a.

FIG. 32c is a top perspective view of another alternative bladder for use with the cartridge mounting plate of FIG. 29a.

FIG. 32d is a bottom perspective view of the bladder of FIG. 32c.

FIG. 32e is a top perspective view of another alternative bladder for use with the cartridge mounting plate of FIG. 29a.

FIG. 32f is a bottom perspective view of the bladder of FIG. 32e.

FIG. 33a is a top plan view of another alternative bladder for use with the cartridge mounting plate of FIG. 29a.

FIG. 33b is a bottom perspective view of the bladder of FIG. 33a.

FIG. 33c is a top plan view of another alternative bladder for use with the cartridge mounting plate of FIG. 29a.

FIG. 33d is a bottom perspective view of the bladder of FIG. 33c.

FIG. 33e is a top plan view of another alternative bladder for use with the cartridge mounting plate of FIG. 29a.

FIG. 33f is an elevational side view of the bladder of FIG. 33e.

FIG. 33g is a top plan view of another alternative bladder for use with the cartridge mounting plate of FIG. 29a.

FIG. 33h is a bottom perspective view of the bladder of FIG. 33g.

FIG. 34a is a wire frame illustration of a vaporizer illustrating in solid view use of the bladder of FIG. 31a.

FIG. 34b is a transparent, top perspective view of a cartridge body including mesh assembly illustrating in solid view use of the bladder of FIG. 31a.

FIG. 34c is a bottom perspective view of the cartridge of FIG. 34b.

FIG. 34d is a bottom perspective view of a cartridge illustrating in solid view use of the bladder of FIG. 31c.

FIG. 34e is a top perspective view of the cartridge of FIG. 34d.

FIG. 35a is a perspective view of an alternative cartridge for use with the vaporizer of FIG. 23 illustrating in solid view a piezoelectric material, mesh material, bladder, and a mechanism for driving fluid from the bladder to the mesh material for aerosolizing.

FIG. 35b is a perspective view of the other side of the cartridge of FIG. 35a.

FIG. 35c is a side elevational view of the cartridge of FIG. 35a.

FIG. 35d is a perspective view of an alternative cartridge for use with the vaporizer of FIG. 23 illustrating in solid view a piezoelectric material, mesh material, bladder, and a mechanism for driving fluid from the bladder to the mesh material for aerosolizing.

FIG. 35e is a view of the cartridge of FIG. 35d without the mechanism of FIG. 35d.

FIG. 35f is another view of the cartridge of FIG. 35e from a side opposite to the side of the view of FIG. 35e.

FIG. 36a is a top perspective view of an alternative cartridge for use with the vaporizer of FIG. 23 illustrating in solid view a piezoelectric material, mesh material, bladder, and a mechanism for driving fluid from the bladder to the mesh material for aerosolizing.

FIG. 36b is a bottom perspective view of the cartridge of FIG. 36a.\

FIG. 36c is a top perspective view of an alternative cartridge for use with the vaporizer of FIG. 23 illustrating in solid view a piezoelectric material, mesh material, bladder, and a mechanism for driving fluid from the bladder to the mesh material for aerosolizing.

FIG. 36d is a bottom perspective view of yet another alternative cartridge for use with the vaporizer of FIG. 23 illustrating in solid view a piezoelectric material, mesh material, bladder, and a mechanism for driving fluid from the bladder to the mesh material for aerosolizing.

FIG. 36e is a top perspective view of the cartridge of FIG. 36d without the mechanism of FIG. 36d.

FIG. 36f is a top perspective view of yet another alternative cartridge for use with the vaporizer of FIG. 23 illustrating in solid view a piezoelectric material, mesh material, bladder, and a mechanism for driving fluid from the bladder to the mesh material for aerosolizing.

FIG. 37a is an elevational view of an alternative cartridge for use with the vaporizer of FIG. 23 illustrating in solid view a bladder thereof.

FIG. 37b is a top perspective view of the cartridge of FIG. 37a.

FIG. 37c is a bottom perspective view of the cartridge of FIG. 37a.

FIG. 37d is a bottom perspective view of yet another alternative cartridge for use with the vaporizer of FIG. 23 illustrating in solid view a bladder thereof which is similar to the bladder of FIG. 37a, but which includes a radial arm for side filling of liquid through injection.

FIG. 37e is a top perspective view of the cartridge of FIG. 37d.\

FIG. 38a is top perspective view of an alternative cartridge for use with the vaporizer of FIG. 23 illustrating in solid view a piezoelectric material, mesh material, and bladder thereof.

FIG. 38b is a bottom perspective view of the cartridge of FIG. 38a.

FIG. 38c is top perspective view of yet another alternative cartridge for use with the vaporizer of FIG. 23 illustrating in solid view a piezoelectric material, mesh material, and bladder thereof.

FIG. 38d is another top perspective view of the cartridge of FIG. 38c.

FIG. 38e is a bottom perspective view of the cartridge of FIG. 38c.

FIG. 39a is a top perspective view of an alternative cartridge for use with the vaporizer of FIG. 23 illustrating in solid view piezoelectric materials, mesh material, and bladder thereof.

FIG. 39b is a bottom perspective view of the cartridge of FIG. 39a.

FIG. 40a is a top perspective view of an alternative cartridge for use with the vaporizer of FIG. 23 illustrating in solid view a piezoelectric material, mesh material, bladder, and foam inserts.

FIG. 40b is a bottom perspective view of the cartridge of FIG. 40a.

FIG. 41 additionally sets forth other potential means for causing the liquid to contact the mesh material, which are shown in contrast to gravity fed systems.

FIG. 42 additionally illustrates four additional low pressure bladder concepts that are contemplated for use in some preferred embodiments of the invention.

FIG. 43 is a schematic diagram of an active ingredient pulmonary delivery nanoparticle in the form of a micelle in accordance with one or more aspects of the invention;

FIG. 44 is a schematic diagram of an active ingredient pulmonary delivery nanoparticle in the form of a liposome carrying an active ingredient within a bilayer in accordance with one or more aspects of the invention; and

FIG. 45 is a schematic diagram of an active ingredient pulmonary delivery nanoparticle in the form of a liposome carrying an active ingredient in a hydrophilic core in accordance with one or more aspects of the invention.

Additional views of many of these cartridges and bladders, and alternative thereof, are disclosed in files of the computer program listing incorporated herein by reference. These files present three-dimensional interactive views using an eDrawing viewer and an Acrobat viewer.

### DETAILED DESCRIPTION

As a preliminary matter, it will readily be understood by one having ordinary skill in the relevant art (“Ordinary Artisan”) that the invention has broad utility and application. Furthermore, any embodiment discussed and identified as being “preferred” is considered to be part of a best mode contemplated for carrying out the invention. Other embodiments also may be discussed for additional illustrative purposes in providing a full and enabling disclosure of the invention. Furthermore, an embodiment of the invention may incorporate only one or a plurality of the aspects of the invention disclosed herein; only one or a plurality of the features disclosed herein; or combination thereof. As such, many embodiments are implicitly disclosed herein and fall within the scope of what is regarded as the invention.

Accordingly, while the invention is described herein in detail in relation to one or more embodiments, it is to be understood that this disclosure is illustrative and exemplary of the invention and is made merely for the purposes of providing a full and enabling disclosure of the invention. The detailed disclosure herein of one or more embodiments is not intended, nor is to be construed, to limit the scope of patent protection afforded the invention in any claim of a patent issuing here from, which scope is to be defined by the claims and the equivalents thereof. It is not intended that the scope of patent protection afforded the invention be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the invention. Accordingly, it is intended that the scope of patent protection afforded the invention be defined by the issued claim(s) rather than the description set forth herein.

Additionally, it is important to note that each term used herein refers to that which the Ordinary Artisan would understand such term to mean based on the contextual use of such term herein. To the extent that the meaning of a term used herein—as understood by the Ordinary Artisan based on the contextual use of such term—differs in any way from any particular dictionary definition of such term, it is intended that the meaning of the term as understood by the Ordinary Artisan should prevail.

With regard to the construction of the scope of any claim in the United States, no claim element is to be interpreted under 35 U.S.C. 112(f) unless the explicit phrase “means for” or “step for” is actually used in such claim element, whereupon this statutory provision is intended to and should apply in the interpretation of such claim element. With regard to any method claim including a condition precedent step, such method requires the condition precedent to be met and the step to be performed at least once but not necessarily every time during performance of the claimed method.

Furthermore, it is important to note that, as used herein, “comprising” is open-ended insofar as that which follows such term is not exclusive. Additionally, “a” and “an” each generally denotes “at least one” but does not exclude a plurality unless the contextual use dictates otherwise. Thus, reference to “a picnic basket having an apple” is the same as “a picnic basket comprising an apple” and “a picnic basket including an apple”, each of which identically describes “a picnic basket having at least one apple” as well as “a picnic basket having apples”; the picnic basket further may contain one or more other items beside an apple. In contrast, reference to “a picnic basket having a single apple” describes “a picnic basket having only one apple”; the picnic basket further may contain one or more other items beside an apple. In contrast, “a picnic basket consisting of an apple” has only a single item contained therein, i.e., one apple; the picnic basket contains no other item.

When used herein to join a list of items, “or” denotes “at least one of the items” but does not exclude a plurality of items of the list. Thus, reference to “a picnic basket having cheese or crackers” describes “a picnic basket having cheese without crackers”, “a picnic basket having crackers without cheese”, and “a picnic basket having both cheese and crackers”; the picnic basket further may contain one or more other items beside cheese and crackers.

When used herein to join a list of items, “and” denotes “all of the items of the list”. Thus, reference to “a picnic basket having cheese and crackers” describes “a picnic basket having cheese, wherein the picnic basket further has crackers”, as well as describes “a picnic basket having crackers, wherein the picnic basket further has cheese”; the picnic basket further may contain one or more other items beside cheese and crackers.

The phrase “at least one” followed by a list of items joined by “and” denotes an item of the list but does not require every item of the list. Thus, “at least one of an apple and an orange” encompasses the following mutually exclusive scenarios: there is an apple but no orange; there is an orange but no apple; and there is both an apple and an orange. In these scenarios if there is an apple, there may be more than one apple, and if there is an orange, there may be more than one orange. Moreover, the phrase “one or more” followed by a list of items joined by “and” is the equivalent of “at least one” followed by the list of items joined by “and”.

Additionally, as used herein unless context dictates otherwise, the following terms have the following meanings.

“Liquid” means a substance that flows freely but is of constant volume, generally having a consistency like that of water (lower viscosity) or oil (higher viscosity). Liquid is generic to and encompasses a solution, a suspension, and an emulsion.

“Solution” means a homogeneous mixture of two or more components. The dissolving agent is the solvent. The substance that is dissolved is the solute. The components of a solution are atoms, ions, or molecules, and the components are usually a nanometer or less in any dimension. An example of a solution is sugar mixed with water.

“Suspension” means a mixture of components that can be evenly distributed by mechanical methods such as shaking or stirring, but that will eventually settle out over an extended period of time. The components in a suspension are generally larger than those in solutions. An example of a suspension is oil mixed with water.

“Colloidal dispersion” means a heterogenous liquid mixture in which a component is dispersed in another component and does not tend to settle out over an extended period of time. The dispersed components generally is larger than components of a solution and smaller than components of a suspension.

“Aerosol” means a colloidal dispersion of a solid or liquid in a gas.

“Emulsion” means a colloidal dispersion of a liquid in a liquid. An example of an emulsion is milk.

“Nanoemulsion” means an emulsion in which the dispersed component comprises nanoparticles.

“Nanoparticle” means a molecule has—or aggregate of molecules have—having no dimension greater than about a micrometer (1,000 nanometers). In accordance with preferred embodiments of aspects and features of the invention, nanoparticles preferably have a dimension of between about 50 and about 200 nanometers.

“Micelle” means a vesicle having a layer of molecules that encapsulate and transport a substance to cells of a body. The encapsulating molecules in a micelle may be surfactants or polymers, for example. A typical micelle in an aqueous solution forms an aggregate with the hydrophilic “head” regions in contact with the surrounding solvent, creating a hydrophobic tail region in the interior of the aggregate.

“Liposome” means a vesicle having at least one bilayer of molecules that encapsulates and transports a substance to cells of a body.

“Microfluidizing machine” means an apparatus that uses microreactor technology to make nanoemulsions through the interaction of liquid streams in defined microchannels. Such technology is described, for example, in U.S. patent application publications 2012/0236680 and 2019/0299171, each incorporated herein by reference. Microfluidizing machines principally utilize high shear forces and impact to emulsify a liquid-liquid system, dispersing one immiscible liquid into another within an interaction chamber. A “Y” chamber preferably is used and may be single-slotted or multi-slotted. Fundamentally, such microreactor technology comprises a large pump that forces a formulation through a very small orifice (i.e., microchannel) at pressures ranging from as low as 3.4 MPa (500 psi) to as high as 275 MPa (40,000 psi). Preferred microfluidizing machines correspond to the processors manufactured, sold, or distributed by Mircofluidics of Newton or Westwood, Massachusetts, under the registered trademark MICROFLUIDIZER, and any and all other apparatus that have the same or equivalent structure for performing the same or equivalent function with the same or equivalent result. The appendix includes a user guide from 2014 for MICROFLUIDIZER processors distributed by Microfluidics, which appendix is incorporated herein by reference.

Referring now to the drawings, as well as to the drawings of the incorporated disclosures of Applicant's other applications, and any U.S. patent application publications thereof and U.S. patents issuing therefrom, one or more preferred embodiments in accordance with one or more aspects and features of the invention are next described. The following description of one or more preferred embodiments is merely exemplary in nature and is in no way intended to limit the invention, its implementations, or uses.

In accordance with electronic devices of the invention, a vibrating mesh is provided for aerosolizing a liquid without smoldering. The aerosolized liquid preferably is in the form of a vapor cloud similar to what a person or observer would surmise to be “vapor” when vaping. In the context of vaping, such preferred devices of the invention therefore are believed to produce an aerosol that is carcinogen free. This is in stark contrast to vaporizers used today to aerosolize e-liquids by heating the e-liquids and desired compounds contained therein (e.g., nicotine) or supplements such as B12, THC/CBD and other drugs or stimulants. As a result of using heating to aerosolize the e-liquids, these vaporizers produce toxic byproducts like formaldehyde, a recognized Group 1 carcinogen for caner, which toxic byproducts then are unfortunately inhaled by a person using the vaporizer. For example, when the liquids are heated, the liquids undergo a thermochemical reaction producing unwanted emissions. The unwanted emissions of the toxic byproducts may cause bodily harm from extended inhalation exposure.

By utilizing a vibrating mesh, preferred electronic devices in accordance with one or more aspects and features of the invention produce an aerosol without using heat and thus advantageously avoid such toxic byproducts created by the vaporizes currently on the market. The electronic devices thereby advantageously produce a carcinogen free aerosol free of harmful emission byproducts.

One of the primary performance metrics evaluated for aerosols is the residual aerodynamic particle size distribution (“APSD”) of the aerosolized drug product. The residual APSD is characterized by the residual mass median aerodynamic diameter (“MMAD”) and the geometric standard deviation (“GSD”). The MMAD signifies the aerodynamic diameter at which half of the aerosolized drug mass lies below the stated diameter.

The MMADR=MMDI×pI×CNV ⅓×pR ⅙, where MMADR (μm) 1 s the mass median aerodynamic diameter of the residual particles, MMDI (μm) is the mass median diameter (MMD) of the initial droplets, CNV (weight fraction) is the concentration of the non-volatile components (e.g., dissolved drug and excipients) in the formulation, and pI and pR are the densities (g/cm3) of the formulation and the residual particles, respectively.

The vibrating mesh may be configured and arranged to produce an aerosol for various applications. For example, the arrangement and geometry of various features of the vibrating mesh, such as the design of the vibrating mesh and more specifically the design of the aperture holes of the vibrating mesh, may be adapted to produce an aerosol with various particle sizes, flow properties, and fine particle fractions. The size (e.g., diameter), shape (e.g., oval, circular, triangular, etc.), spacing (e.g., distance between aperture holes, aperture hole density), etc. of the aperture holes may be configured and modified to adjust the size of the aerosol particles for specific applications. Additionally, the thickness of the mesh, especially when in the form of a plate, may also be configured to optimize aerosol properties. For example, the thickness of the plate may impart different properties and characteristics to the aerosol. Depending on the thickness of the plate, the holes may taper with a chamfer such that the entrance and/or exit diameter is larger than the bore diameter of the aperture hole. In another example, the aperture holes may have a constant diameter without a taper.

In another example, the rigidity of the mesh assembly may be configured to prevent oscillations of varying amplitude across the surface of the mesh, which could result in inconsistent aerosolization performance. For example, the thickness, geometry, and material selection for the vibrating mesh material may enhance the rigidity to prevent unwanted oscillations thereof. In some embodiments, the mesh material may be constructed from a metal alloy, to provide adequate rigidity, mass, durability and inert chemical properties for the aerosolization of different drug formulations. Indeed, the design and dimensions of the mesh material may be selected to optimize the device based on the intended application or use case. For example, the vibrating mesh may be configured to adjust the MMADR, fine particle fraction, air/particle velocity, etc. Additionally, the mesh material may also determine the resulting particle properties such as volume diameter, bulk density, tap density, shape, charge, etc.

In addition to the mechanical aspects of the mesh material and its operation, it is believed that the material substrate from which the mesh is constructed and the way in which the holes are generated have important implications for the aerosolization of different drug formulations. In some embodiments, the aperture holes may be electro formed or laser formed. It should be appreciated that other manufacturing methods may be used to form the aperture holes. Example methods for mesh production include electroplating and laser cutting, which may be used to produce a tapered hole. A tapered hole may optimize mesh performance by amplifying flow at the nozzle while reducing viscose losses. The electroplating method makes use of a lithographic plate and the eventual size of the mesh holes may be determined by the duration of the electroplating process. The holes become smaller as the metal is deposited on the edge of the hole over time. Laser cutting involves the use of a laser beam to cut the mesh holes into a thin sheet of metal or polymer material. Laser cutting metal may result in molten material being deposited around the hole, which may be removed by polishing.

In some embodiments, the liquid delivery system may be adapted for a specific liquid. For example, viscosity may be a controlling variable in the size of the aperture holes of the vibrating mesh. Some preferred liquids comprise nicotine, which is less viscous than a cannabinoid derivative (e.g., tetrahydrocannabinol (“THC”) and cannabidiol (“CBD”)), which has a higher viscosity. Other considerations may include water solubility, surface tension, acidity and/or basicity, and whether the liquid contains a liquid carrier. Some preferred liquids indeed comprise liquid carriers and, in particular, liposomal carriers. Various liquids and formulations may be used to form aerosols from electronic devices of the invention. These formulations may have widely different physiochemical properties, such as surface tension, density, viscosity, characteristics of intramolecular forces within the formulation and whether the formulation is a pure liquid or a suspension of particles within a liquid. Each of the above-mentioned physiochemical properties may affect the functionality, consistency, efficacy, and end properties of the resulting aerosol or vapor cloud.

The liquid delivery system also may be designed to provide different flow rates. For example, the pump may be an active pump or a passive pump. Additionally, in some preferred embodiments the output rate, pressure supplied by the pump, or both, may be adjusted to provide different flow rates.

In some embodiments, the geometry of the mesh may be the form of a dome-like structure. In some embodiments, the mesh may be flat and may be in the form of a plate. Other orientations and geometries also are contemplated within the scope of the invention.

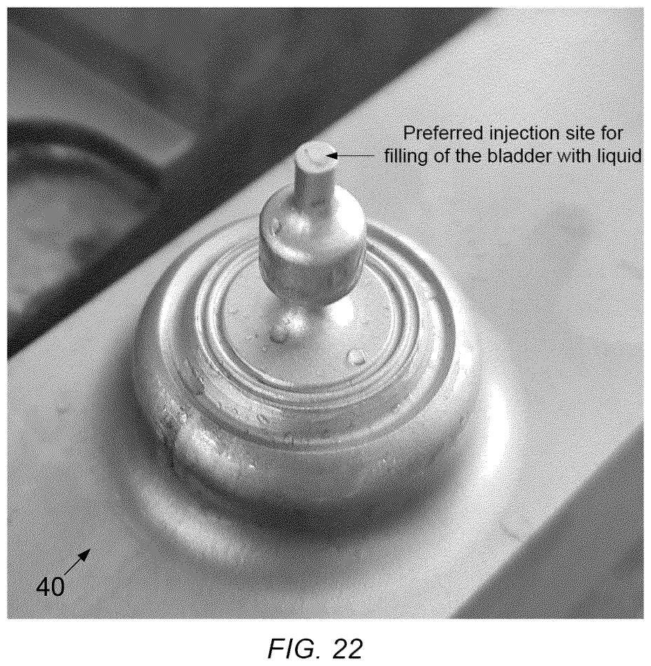

Additionally, in electronic devices of the invention, the vibrating mesh assembly may include a single layer oscillating piezoelectric material to aerosolize the liquid. In an example, the mesh assembly may have a double or multi-layer structure, and multiple mesh membranes may be arranged to induce an optimum MMAD and/or APSD for the aerosolized liquid. A plurality of vibrating meshes also may be used in the mesh assembly in some embodiments; FIG. 22 for example illustrates a mesh assembly that includes two separate vibrating meshes spaced apart from one another.

Additionally, the mesh assembly may be constructed from one or more different piezoelectric materials to optimize the MMAD and/or APSD.

Additionally, the arrangement and design of the mesh assembly (e.g., placement of the holes, angstrom size) and hygroscopic effects of the lungs may be considered for optimum deposition and diffusion into the bloodstream.

In some embodiments, the electronic device is configured to create a fine particle low velocity aerosol. The resulting aerosol or vapor cloud may be configured to reduce or soften the potential irritation of the airways and lungs. In some embodiments, the encapsulation techniques may create the ideal person experience. As mentioned above, the lungs have clearance mechanisms to prevent invasion of unwanted airborne particles from entering the body. To ensure that the fine particle, low velocity aerosol that achieves central and deep lung deposition, the electronic device and/or formulation may be adapted such that an aerosol is produced that eludes the lung's various lines of defense.

For example, progressive branching and narrowing of the airways encourage impaction of particles. Larger the particle sizes, greater velocities of incoming air, and more abrupt bend angles of bifurcations and the smaller the airway radius increase the probability of deposition by impaction. In essence, the end person may sense/feel more or less impaction based on the above parameters.

Additionally, the lung has a relative humidity of approximately 99.5%. The addition and removal of water can significantly affect the particle size of a hygroscopic aerosol and thus deposition itself. Drug particles are known to be hygroscopic and grow or shrink in size in high humidity, such as in the lung. A hygroscopic aerosol that is delivered at relatively low temperature and humidity into one of high humidity and temperature may increase in size when inhaled into the lung. For example, the rate of growth may be a function of the initial diameter of the particle. As it relates to size and diameter, particles may be deposited by inertial impaction, gravitational sedimentation or diffusion (Brownian motion) depending on their size. While deposition occurs throughout the airways, inertial impaction usually occurs in the first ten generations of the lung, where air velocity is high and airflow is turbulent.

In the therapeutic/medical environment, most particles larger than 10 micrometers are deposited in the oropharyngeal region with a large amount impacting on the larynx, particularly when the drug is inhaled from devices requiring a high inspiratory flow rate (e.g., as with dry powder inhalers (“DPIs”)) or when the drug is dispensed from a device at a high forward velocity. The large particles are subsequently swallowed and contribute minimally, if at all, to the therapeutic response. In the tracheobronchial region, inertial impaction may also play a significant role in the deposition of particles, particularly at bends and airway bifurcations. Deposition by gravitational sedimentation may typically predominate in the last five to six generations of airways (smaller bronchi and bronchioles), where air velocity is low. Due to the low velocity, large volume aerosol that is produced in accordance with preferred embodiments of the invention, the aerosol may be less irritating to a person.

In the alveolar region, air velocity is typically negligible, and thus the contribution to deposition by inertial impaction is typically nonexistent. Particles in this region may have a longer residence time and may be deposited by both sedimentation and diffusion. Particles not deposited during inhalation may be exhaled. Deposition due to sedimentation affects particles down to 0.5 micrometers in diameter, whereas below 0.5 micrometers, the main mechanism for deposition is by diffusion.

Targeting the aerosol to conducting or peripheral airways may be accomplished by altering the particle size of the aerosol and/or the inspiratory flow rate. For example, aerosols with a MMAD of approximately 5 micrometers to 10 micrometers may be deposited in the large conducting airways and oropharyngeal region. Particles ranging from approximately 1 micrometer to 5 micrometers in diameter may be deposited in the small airways and alveoli with more than 50% of the particles having a diameter of three micrometers being deposited in the alveolar region.

In some embodiments, the electronic device includes a piezoelectric crystal that vibrates at a high frequency when electrical current is applied. In some embodiments, the vibration may be in the range of 0.5 to 5.0 MHz, and more specifically within the range of 1.2 to 2.4 MHz. The vibration of the crystal is transmitted to a transducer horn that is in contact with the liquid to be aerosolized. Vibrations transmitted by the transducer horn cause upward and downward movement of a mesh in the form, for example, of a plate, and the liquid passes through the apertures in the mesh plate to form an aerosol. In some embodiments, the mesh plate consists of a plurality of tapered holes (e.g., 500 holes; 1,000 holes; 6,000 holes). Each tapered hole may have a diameter of approximately 3 micrometers. In other examples, larger or smaller diameters may be appropriate for different liquids or applications. The aperture holes advantageously amplify the vibration of the transducer horn throughout the liquid and reduce the amount of power required to generate the aerosol. For example, using a low frequency of vibration with a mesh plate containing numerous minute holes allows efficient generation of a fine particle mist.

In some embodiments, aqueous liquids may be more suitable to generating an aerosol with electronic devices of the invention when compared to other more viscous liquids. In some embodiments, the aqueous liquids may include ethanol, which itself may be a primary liquid carrier of the liquid.

Additionally, in some preferred embodiments ultrasonicated a liposomal nanoemulsions comprises the liquid carrier of the liquid delivery system. Nanoemulsions may be sonicated where liposomes work as carriers for active agents. In some embodiments, liposomes may be prepared and formed (e.g., by ultrasound) for the entrapment of active agents. In some instances, emulsifiers are added to the liposomal dispersions to stabilize higher amounts of lipids; however, additional emulsifiers may cause a weakening on the barrier affinity of a liquid (e.g., phosphatidylcholine). Nanoparticles (e.g., nanoparticles composed of phosphatidylcholine and lipids) preferably are used to solve this. Thus, in some embodiments, nanoparticles are used that preferably are formed by an oil droplet that is covered by a monolayer of phosphatidylcholine. It is believed that the use of nanoparticles allows formulations which are capable of absorbing more lipids and which remain stable whereby additional emulsifiers may not be needed.

As discussed above, ultrasonication is a method for the production of nanoemulsions and nanodispersions. In some embodiments, an intensive ultrasound supplies the power needed to disperse a liquid phase (dispersed phase) in small droplets in a second phase (continuous phase). In the dispersing zone, imploding cavitation bubbles cause intensive shock waves in the surrounding liquid and result in the formation of liquid jets of high liquid velocity. In order to stabilize the newly formed droplets of the disperse phase against coalescence, emulsifiers (surface active substances, surfactants) and stabilizers are added to the emulsion. As coalescence of the droplets after disruption influences the final droplet size distribution, efficiently stabilizing emulsifiers may be used to maintain the final droplet size distribution at a level that is equal to the distribution immediately after the droplet disruption in the ultrasonic dispersing zone.

Some liposomal dispersions (e.g., those based on unsaturated phosphatidylcholine) may lack in stability against oxidation. The stabilization of the dispersion can be achieved by antioxidants, such as by a complex of vitamins C and E. For example, the entrapment of the essential oil in liposomes may increase the oil stability.

In some embodiments, the vibrating mesh is configured to create a fine particle low velocity aerosol which is well suited for central and deep lung deposition. By producing a fine particle, low velocity aerosol, one or more preferred electronic devices of the invention advantageously can produce an aerosol that is adapted to target small airways in the management of asthma and COPD.

Additionally, some embodiments, a pump system is utilized to pump or push the liquid to be aerosolized into contact with the vibrating mesh whereby droplets of the liquid are created on the other side of the vibrating mesh on the order of 1 to 4 microns. While it is contemplated that a capillary pump may be used (wherein the liquid is drawn into contact with the mesh material through capillary action), electronic devices of the invention also may preferably comprise a pump system that is powered by an electrical power source of the device, such as batteries and, preferably, rechargeable batteries. Such a pump system preferably comprises a piezoelectric motor. In some embodiments, however, an active pump system is not used, and the liquid may be gravity-fed to a vibrating mesh or other vibrating structure. Thus, a gravitational pump may be used in such embodiments. This is particularly contemplated when an electronic device of the invention is used in a generally upright position as a nebulizer for drug delivery. In most preferred embodiments, however, the electronic device is orientation-agnostic and generally works as intended in any orientation relative to the directional forces of gravity.

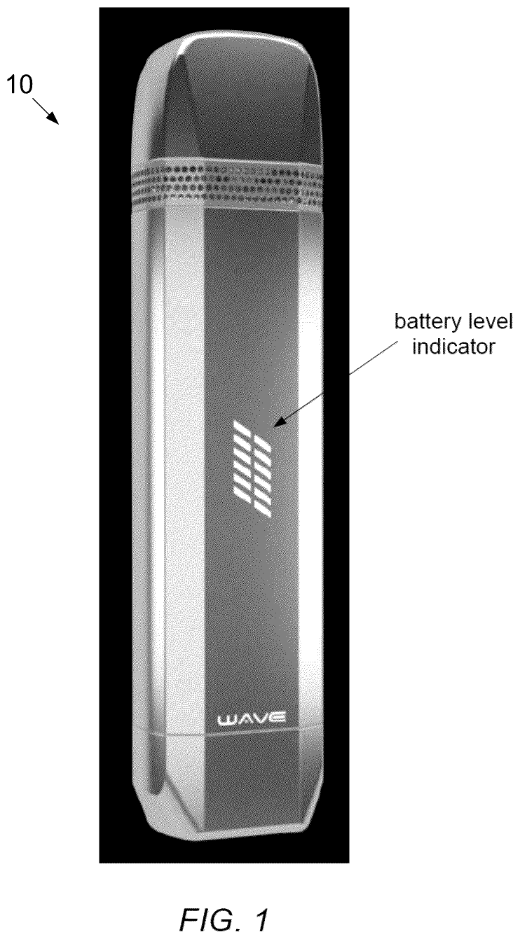

Turning now to the drawings, FIGS. 1-8, a preferred embodiment of an electronic device in the form of a “vaporizer” is illustrated in accordance with one or more aspect and features of the invention. Other forms of an electronic device in accordance with the present include vapes, vape pens, and nebulizers. Other terminology may be given to electronic devices of the present invention. In any event, electronic devices of the present invention produce an aerosol for inhalation whatever commercial or consumer name may be given.

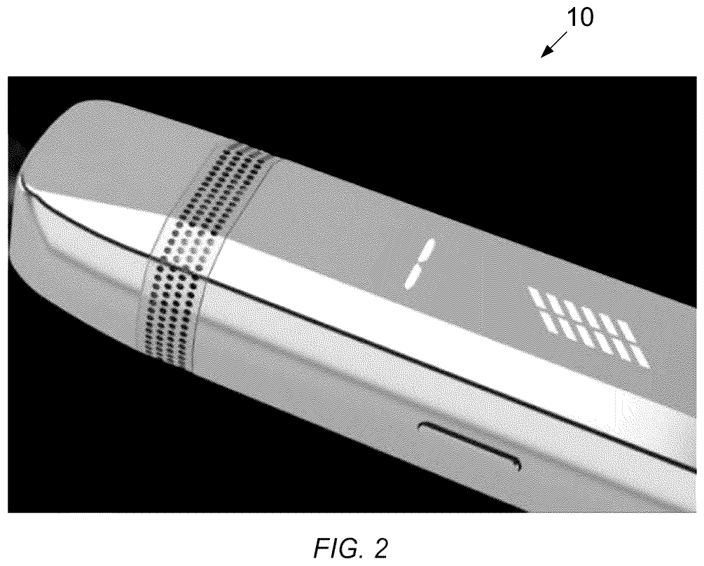

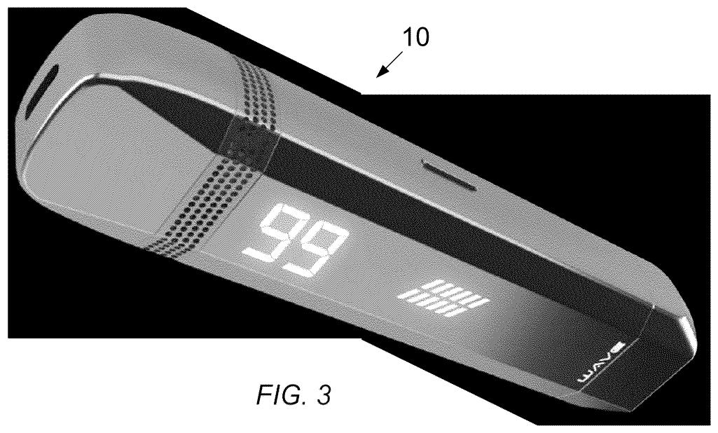

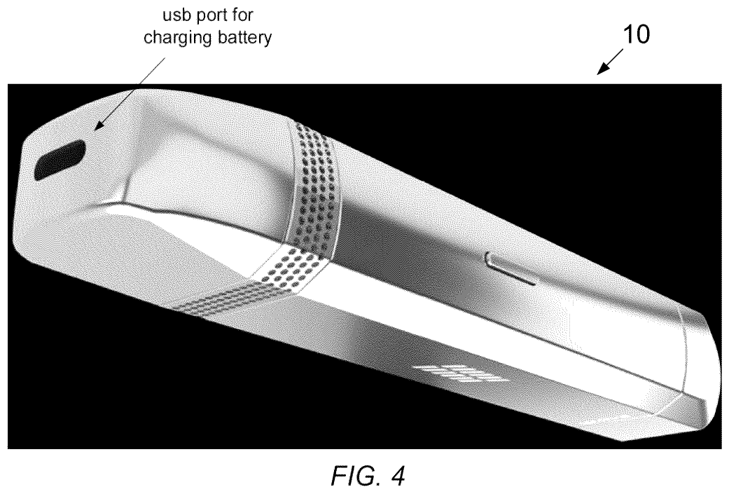

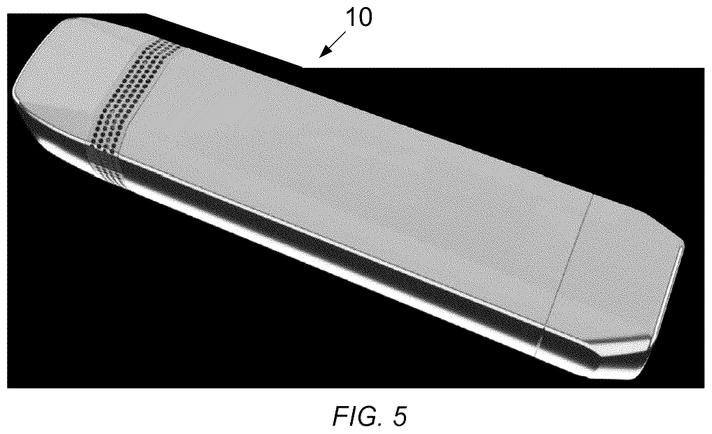

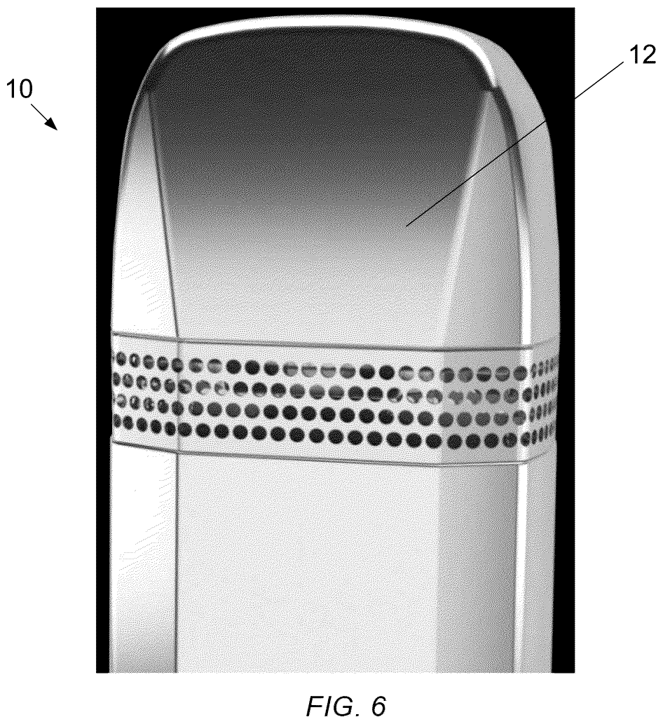

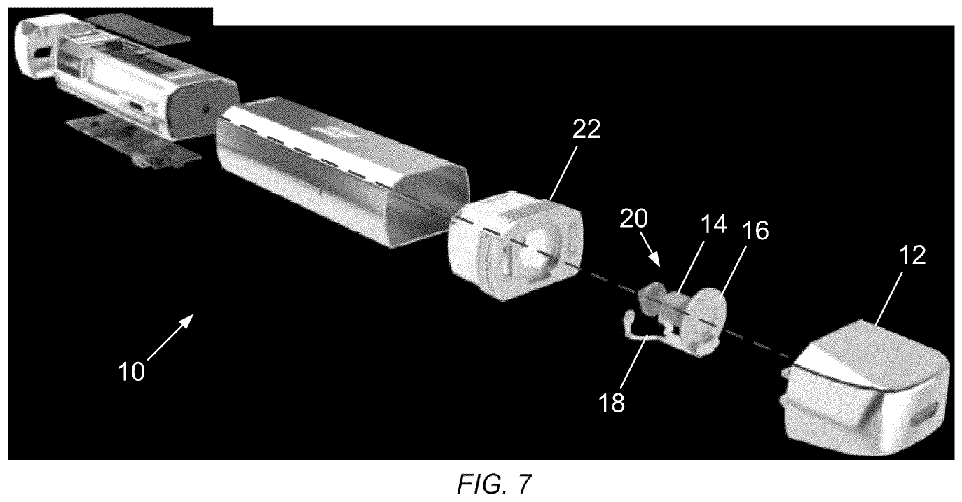

Specifically, FIG. 1 is a perspective view of a preferred embodiment of a vaporizer 10 in accordance with one or more aspect and features of the invention; FIG. 2 is a partial view of the vaporizer 10 of FIG. 1 showing in closeup a counter, battery indicator, and mouthpiece thereof; FIG. 3 is another perspective view of the vaporizer 10 of FIG. 1; FIG. 4 is still yet another perspective view of the vaporizer 10 of FIG. 1; FIG. 5 is a perspective view of the other side of the vaporizer 10 seen in FIG. 1; FIG. 6 is a perspective view of one of two opposite ends of the vaporizer 10 of FIG. 1, which illustrated end comprises the mouthpiece 12 of the vaporizer; and FIG. 7 is an exploded view of the vaporizer 10 of FIG. 1. The bladder 14 can be seen illustrated in blue with the piezo mesh disk 16 and electrical contacts 18 attached to form the mesh assembly 20. The mesh assembly is retained within a body of the cartridge 22, which cartridge is seen to have a perforated band in FIG. 7.

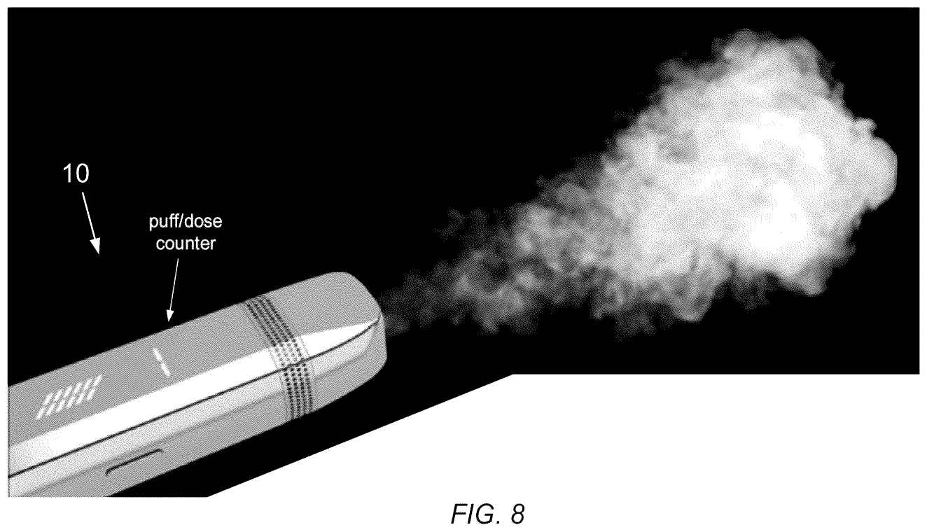

Furthermore, FIG. 8 is a vapor cloud that is produced by a push of the button of the vaporizer 10 of FIG. 1, which vapor cloud preferably has a known quantity of the substance to be inhaled per push of the button/aerosolizing cycle of the vaporizer.

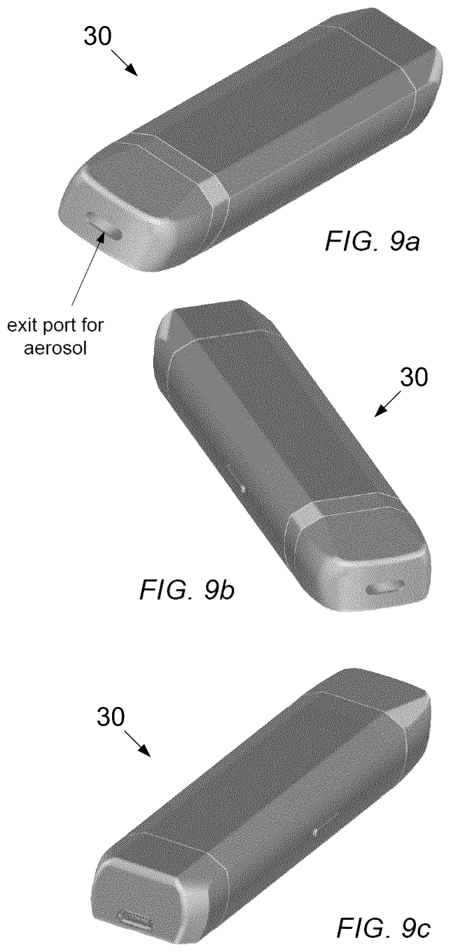

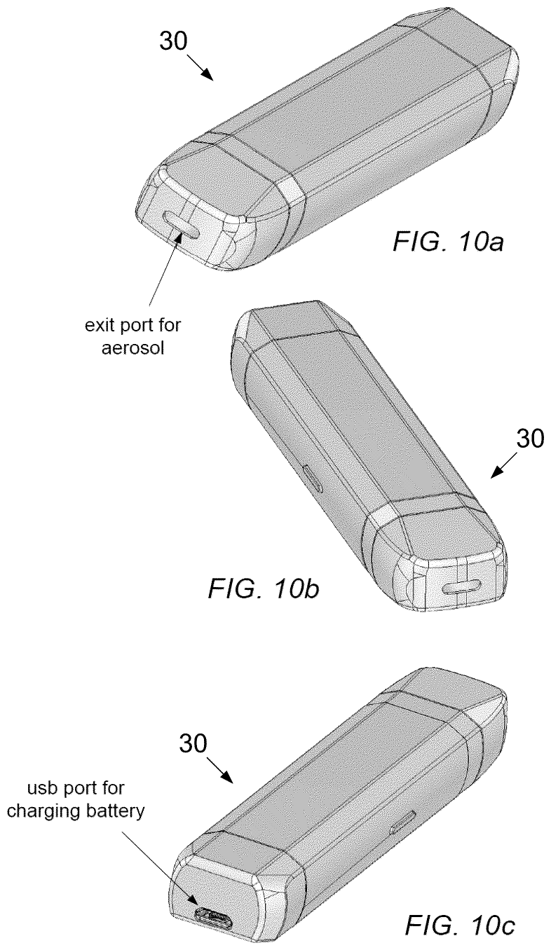

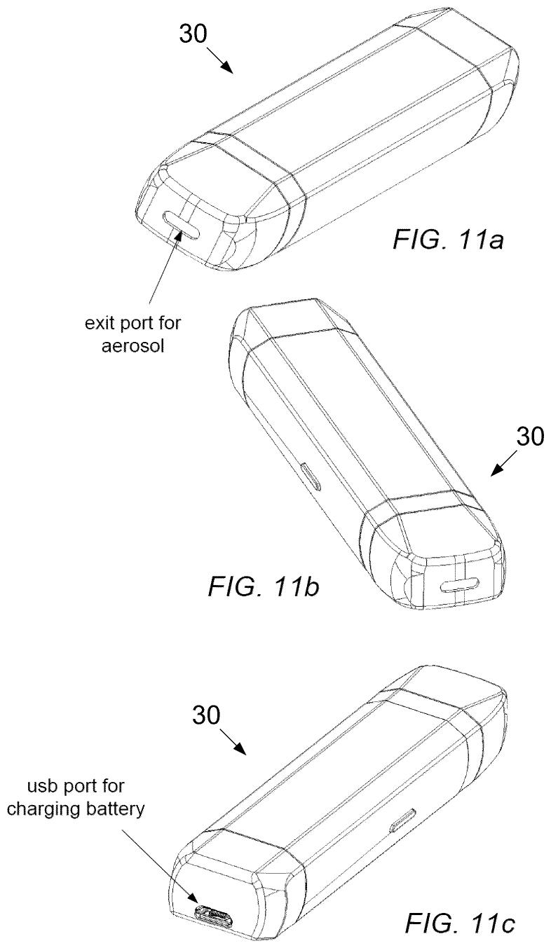

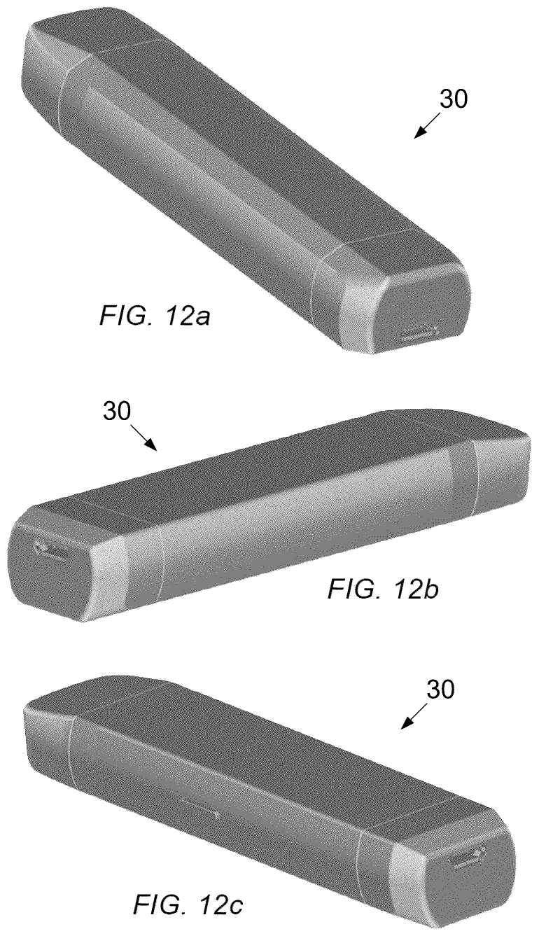

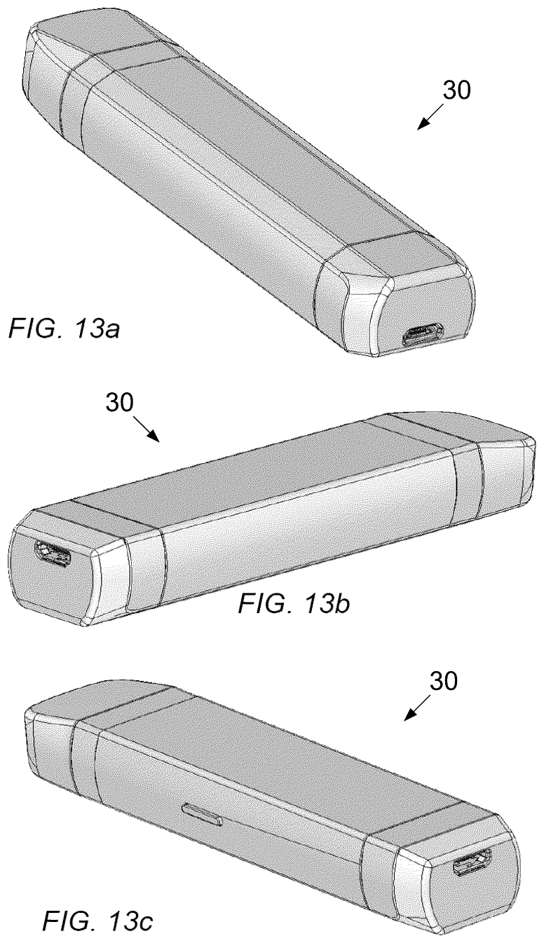

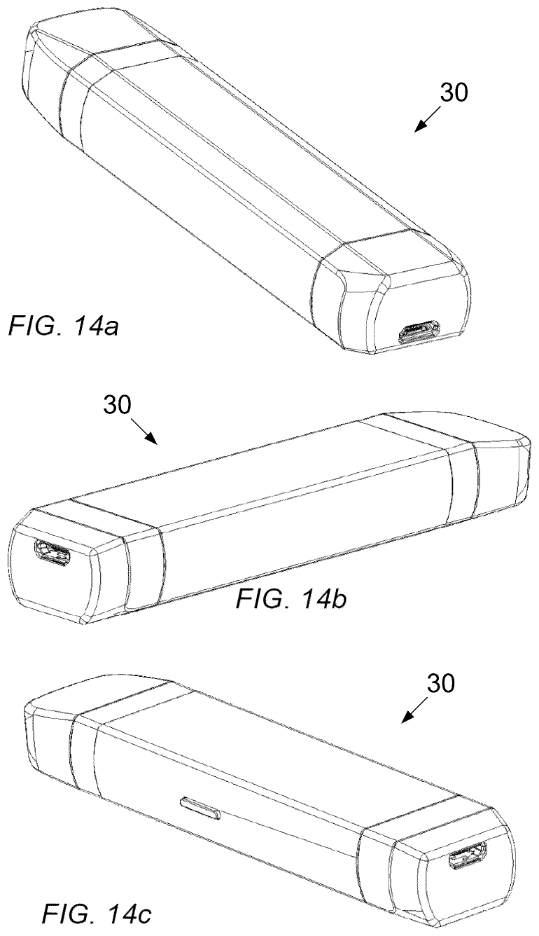

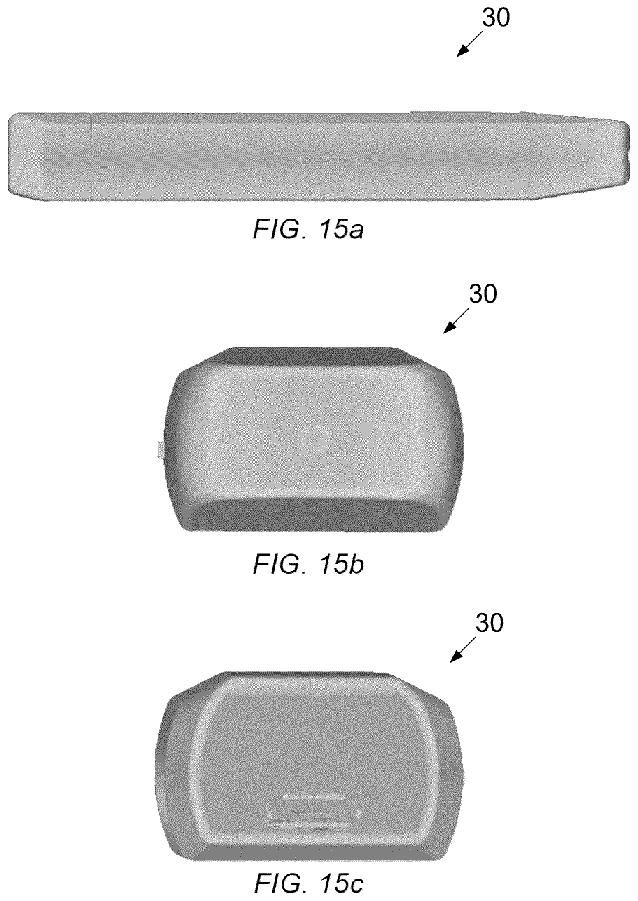

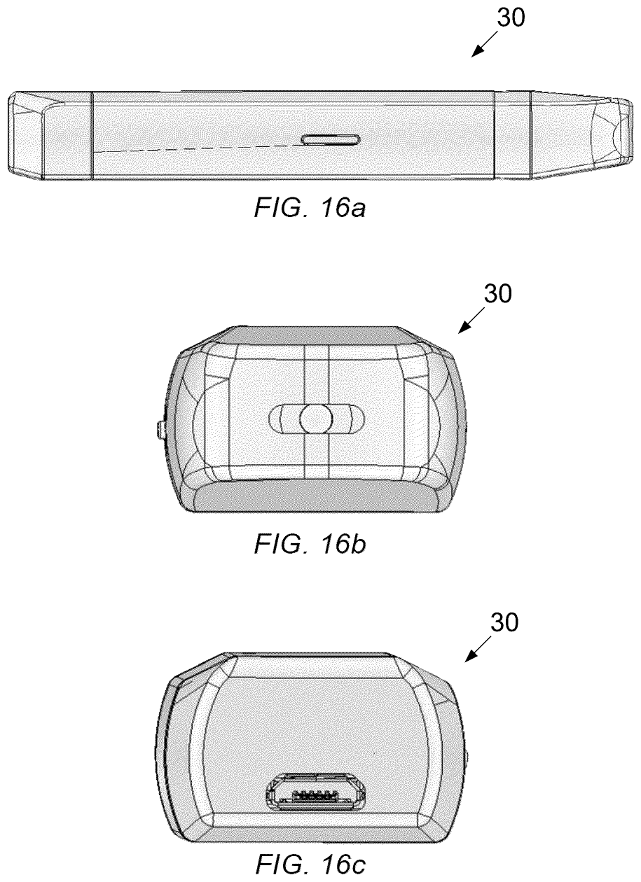

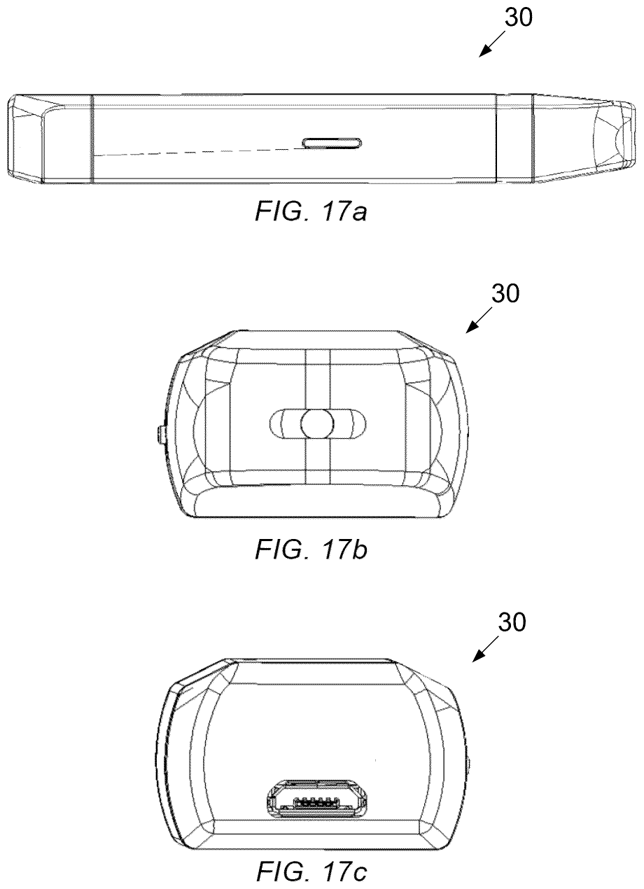

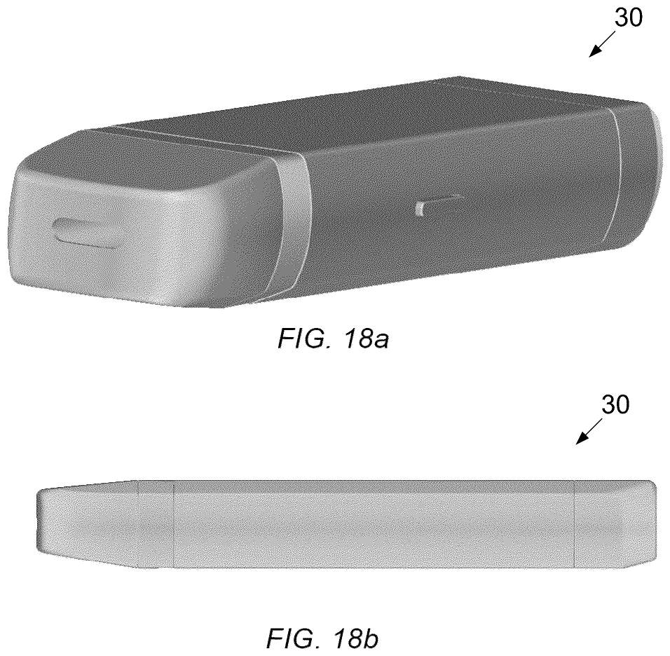

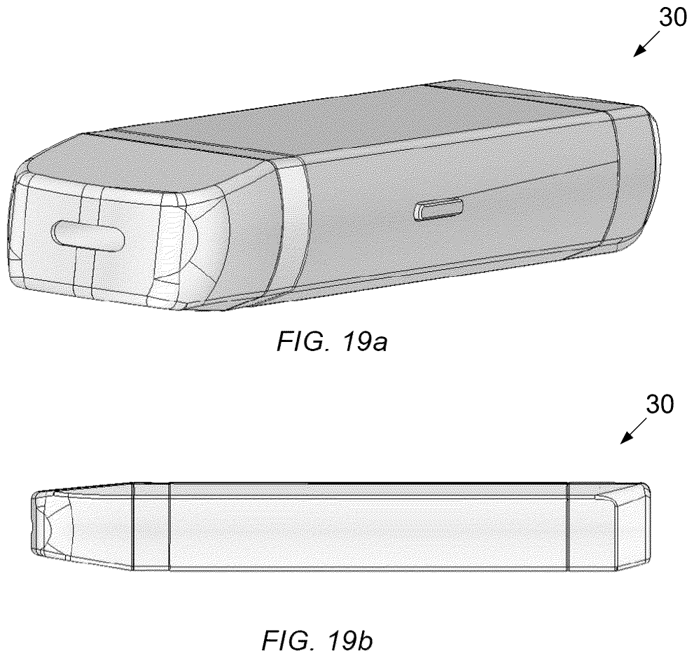

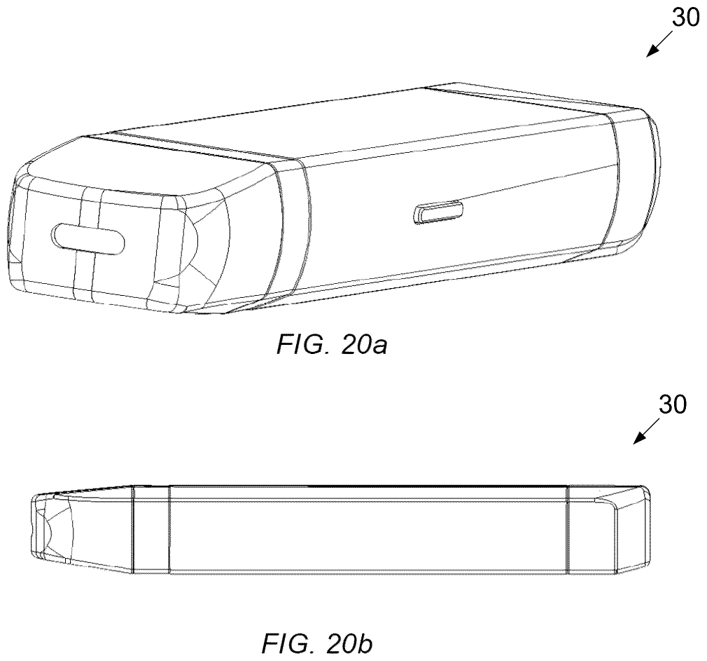

Another preferred embodiment of an electronic device in the form of a vaporizer 30 is illustrated in FIGS. 9a-20b. Specifically, FIG. 9a is a solid, perspective view of an end of a second preferred embodiment of a vaporizer in accordance with one or more aspects and features of the invention; FIG. 9b is another solid, perspective view of the vaporizer of FIG. 9a; FIG. 9c is a solid, perspective view of an end of the vaporizer opposite to the end shown in FIGS. 9a and 9b; FIG. 10a is a solid line drawing of the view seen in FIG. 9a; FIG. 10b is a solid line drawing of the view seen in FIG. 9b; FIG. 10c is a solid line drawing of the view seen in FIG. 9c; FIG. 11a is a line drawing of the view seen in FIG. 9a; FIG. 11b is a line drawing of the view seen in FIG. 9b; FIG. 11c is a line drawing of the view seen in FIG. 9c; FIG. 12a is a solid, perspective view of the opposite end of the second preferred embodiment, which end is the subject of focus in FIG. 9c; FIG. 12b is another solid, perspective view of the end of the vaporizer of FIG. 12a; FIG. 12c is another solid, perspective view of the end of the vaporizer of FIG. 12a; FIG. 13a is a solid line drawing of the view seen in FIG. 12a; FIG. 13b is a solid line drawing of the view seen in FIG. 12b; FIG. 13c is a solid line drawing of the view seen in FIG. 12c; FIG. 14a is a line drawing of the view seen in FIG. 12a; FIG. 14b is a line drawing of the view seen in FIG. 12b; FIG. 14c is a line drawing of the view seen in FIG. 12c; FIG. 15a is a solid, perspective view of a side of the vaporizer of FIG. 12a, which side includes the button; FIG. 15b is a solid, plan view of a top end of the vaporizer of FIG. 12a; FIG. 15c is a solid, plan view of the bottom end of the vaporizer of FIG. 12a; FIG. 16a is a solid line drawing of the view seen in FIG. 15a; FIG. 16b is a solid line drawing of the view seen in FIG. 15b; FIG. 16c is a solid line drawing of the view seen in FIG. 15c; FIG. 17a is a line drawing of the view seen in FIG. 15a; FIG. 17b is a line drawing of the view seen in FIG. 15b; FIG. 17c is a line drawing of the view seen in FIG. 15c; FIG. 18a is a solid perspective view of the vaporizer of FIG. 12a; FIG. 18b is another solid side view of the vaporizer of FIG. 12a; FIG. 19a is a solid line drawing of the view seen in FIG. 18a; FIG. 19b is a solid line drawing of the view seen in FIG. 18b; FIG. 20a is a line drawing of the view seen in FIG. 18a; and FIG. 20b is a line drawing of the view seen in FIG. 18b.

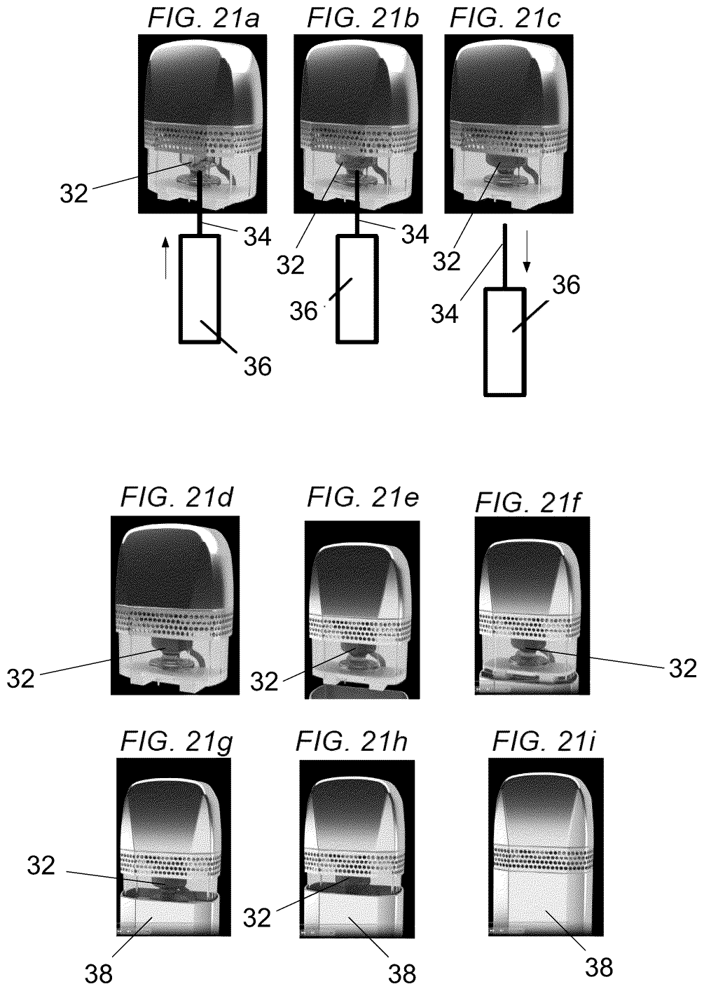

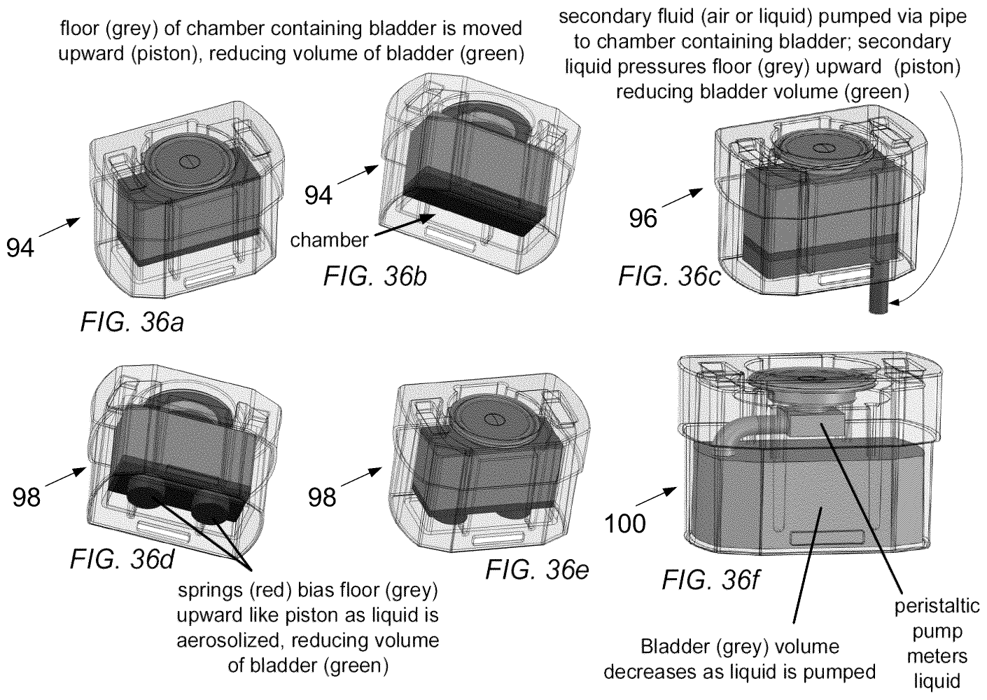

FIGS. 21a-21c collectively illustrate filling of a bladder 32 of the cartridge after the bladder has been installed in the cartridge by injecting fluid directly into the bladder using a needle 34 of an injector 36. Thereafter, the cartridge is secured to the main body chassis 38 of the vaporizer, as illustrated in FIGS. 21d-21i. This may be accomplished by an end-user when replacing a depleted cartridge with a new cartridge included in a pack of disposable cartridges purchased by the user, or during assembly of a vaporizer for sale to a user during manufacture and assembly of the vaporizer. The injection site when filling the bladder preferably is at a distal end of the bladder relative to a mouth of the bladder where a liquid is maintained in contact with the mesh material; however, alternative injection sites are contemplated. Indeed, the bladder of FIGS. 37d and 37e illustrates a radial arm by which the bladder is filled from a side of the bladder rather than bottom of the bladder.

FIGS. 21d-21i collectively illustrate mounting of the cartridge to a base of the vaporizer of FIGS. 1-8.

FIG. 22 is a perspective view of a preferred embodiment of a self-healing, silicone bladder 40 after injection molding thereof in accordance with one or more aspects and features of the invention. The bladder of FIG. 22 has a capacity of about 2.5 milliliters. In other embodiments, the volume of the bladder is as much as 0.35 milliliters.

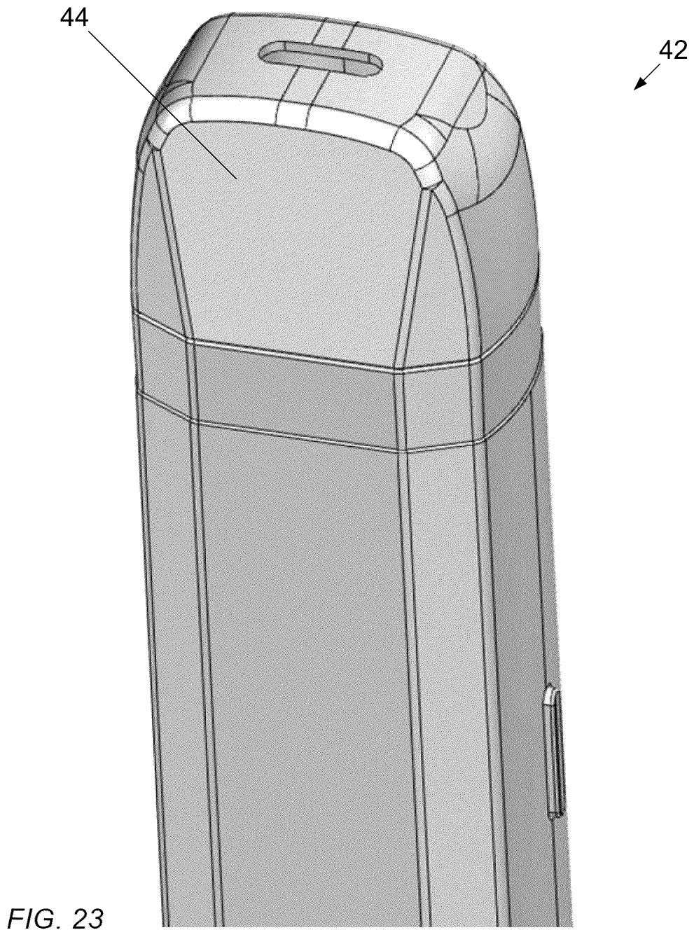

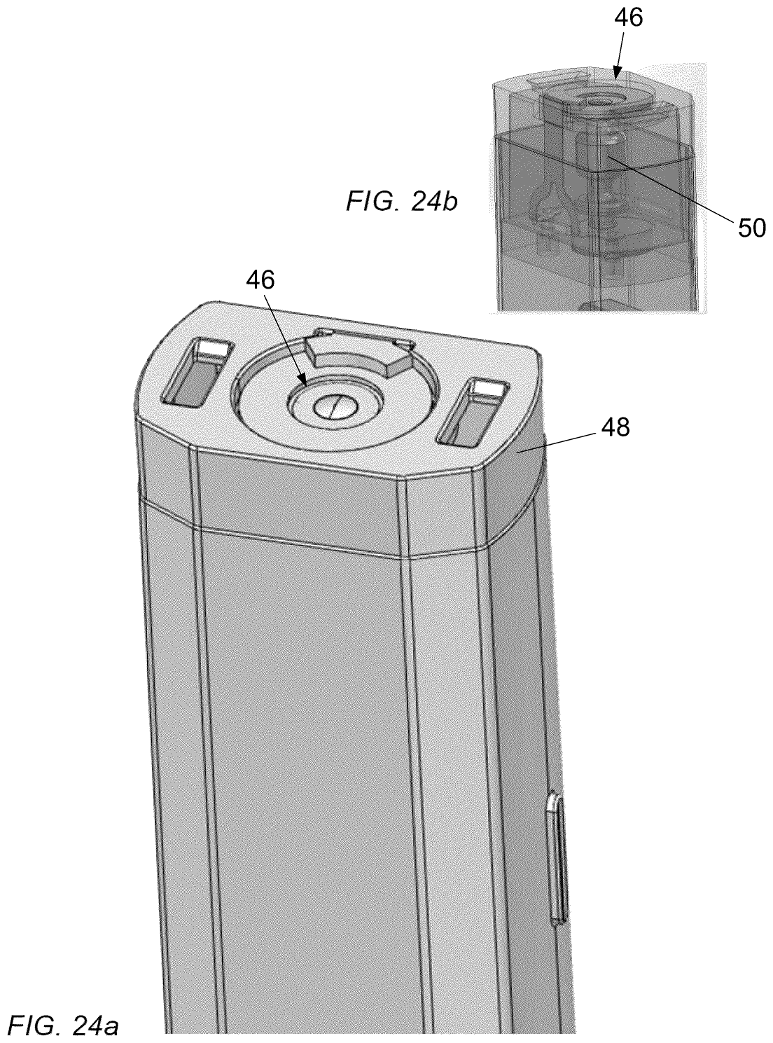

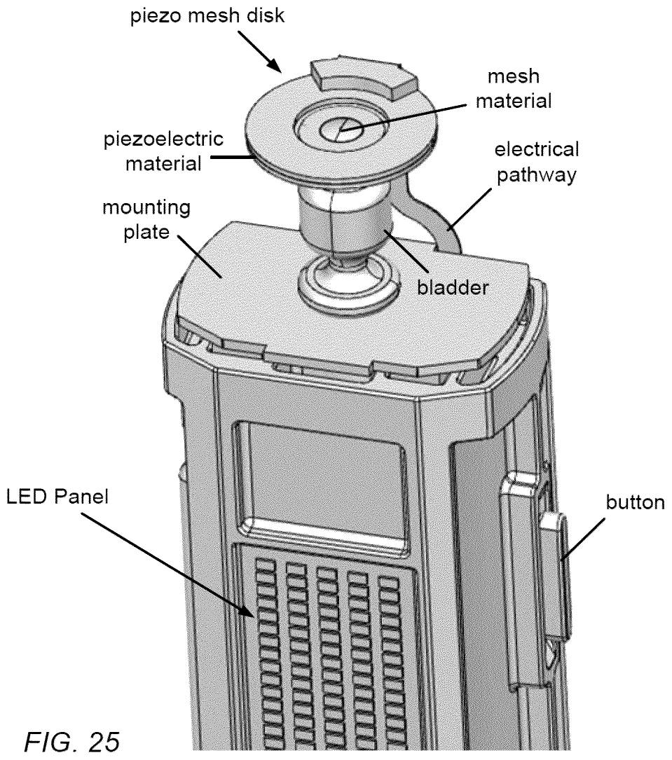

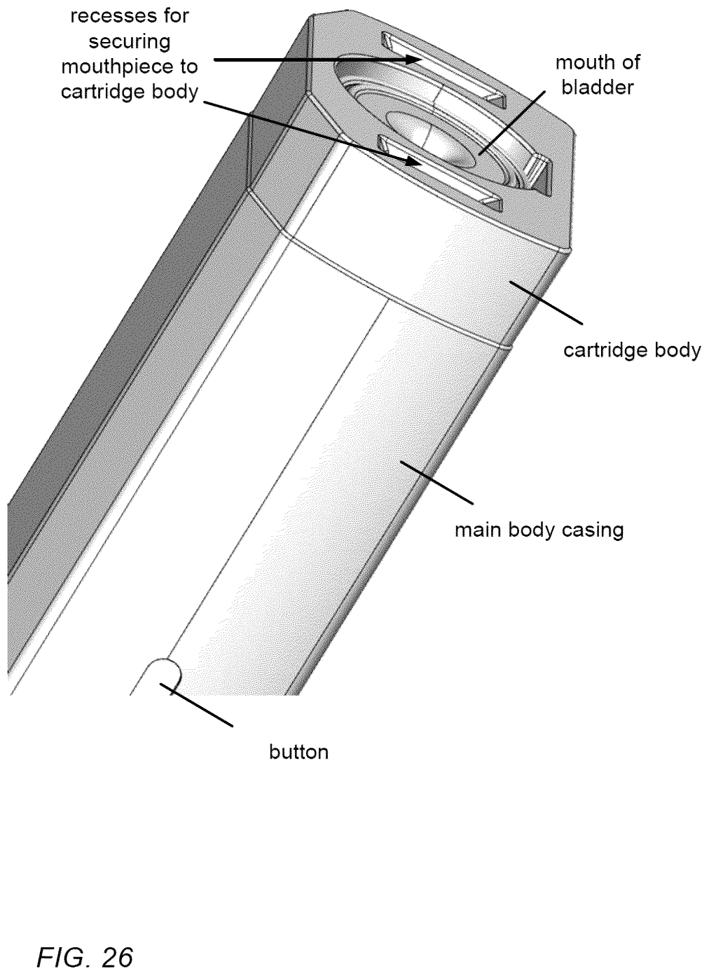

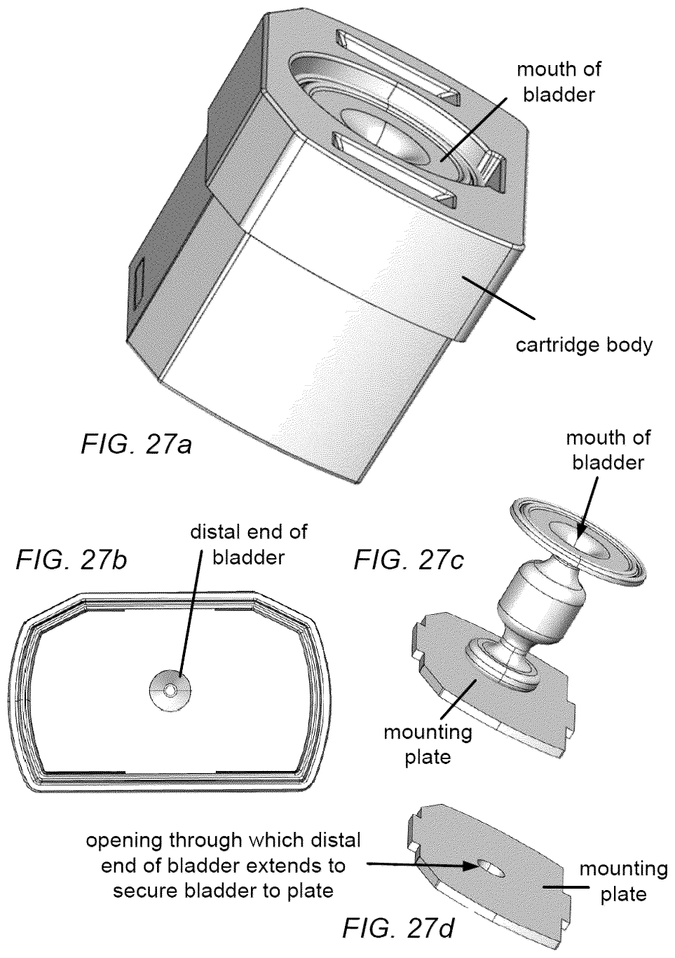

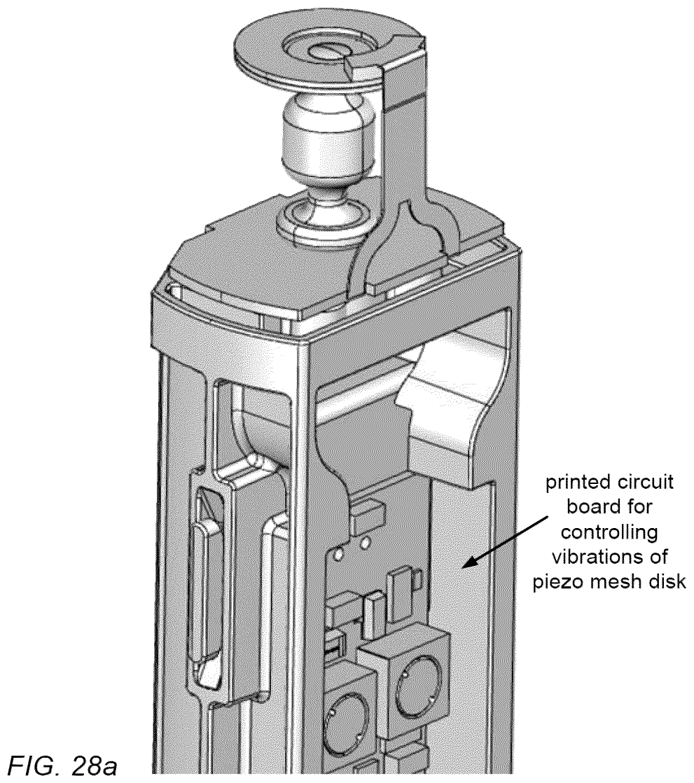

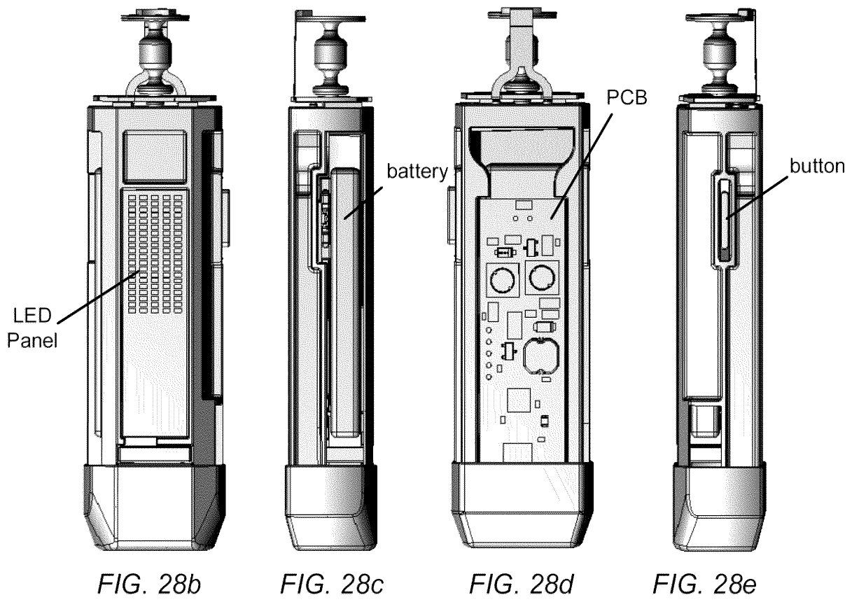

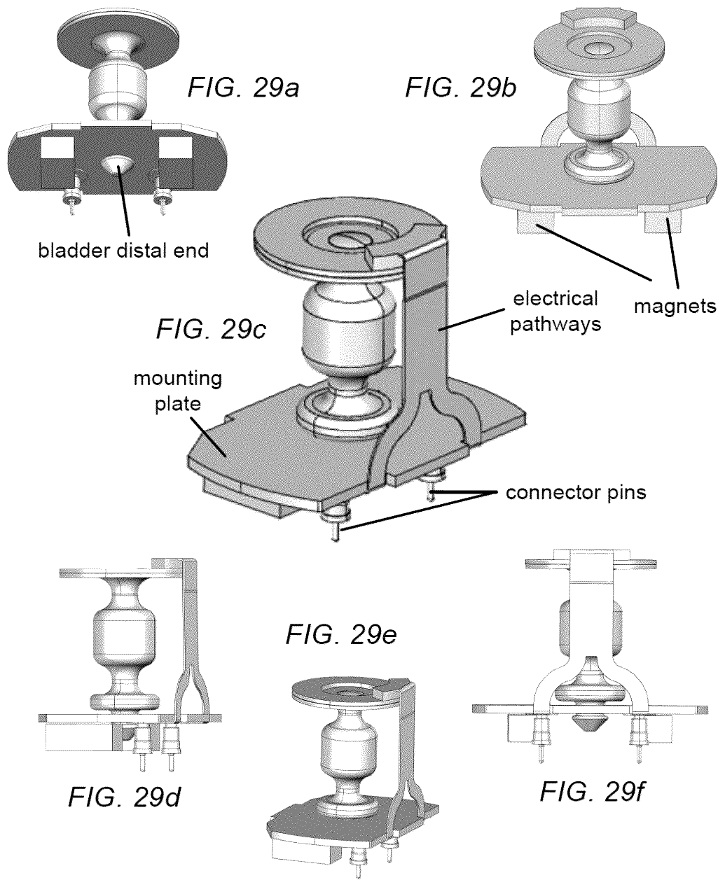

A third preferred embodiment of an electronic device in the form of a vaporizer 42 is illustrated in FIGS. 23-30i. In particular, FIG. 23 is a partial perspective view of an end of a preferred embodiment of a vaporizer in accordance with one or more aspects and features of the invention, which end comprises a mouthpiece 44 of the vaporizer; FIG. 24a is a view of the vaporizer as seen in FIG. 23 wherein the mouthpiece has been removed to reveal a piezo mesh disk 46. As seen in FIG. 24a, the piezo mesh disk is received with a cartridge body 48; FIG. 24b is a transparent view of the vaporizer as seen in FIG. 24a, which reveals a bladder 50 and the mesh assembly including the piezo mesh disk contained within the cartridge body in accordance with one or more aspects and features of the invention; FIG. 25 is a perspective front view of the end of the vaporizer as seen in FIG. 24a, wherein the cartridge body and a main body casing have been removed to reveal the bladder secured to a mounting plate of the cartridge that, in turn, is secured to a main body chassis of the vaporizer. An LED panel secured to the main body chassis of the vaporizer also is revealed in FIG. 25. The main body casing preferably is translucent, at least in the area covering and extending over the LED panel, whereby lighting from the LED panel passes through the main body casing for reading of the LED display but whereby the LED panel itself is otherwise concealed and hidden from sight, as represented for example in FIG. 3; FIG. 26 is another view of the vaporizer as seen in FIG. 24a, wherein the piezo mesh disk has been removed to reveal a mouth of the bladder; FIG. 27a is another view of the vaporizer as seen in FIG. 26, wherein just the cartridge body and bladder are shown; FIG. 27b is a bottom plan view of the cartridge as seen in FIG. 27a; FIG. 27c is a perspective view of the bladder of the cartridge of FIG. 27a, which bladder is seen secured to the cartridge mounting plate; FIG. 27d is a perspective view of just the cartridge mounting plate as seen in FIG. 27c; FIG. 28a is a perspective back view of the vaporizer as seen in FIG. 25; FIG. 28b is an elevational front view of the vaporizer as seen in FIG. 28a; FIG. 28c is an elevational first side view of the vaporizer as seen in FIG. 28a; FIG. 28d is an elevational back view of the vaporizer as seen in FIG. 28a; FIG. 28e is an elevational second side view of the vaporizer as seen in FIG. 28a; FIG. 29a is a bottom perspective view of the bladder and mesh assembly, the cartridge mounting plate, and magnets of the cartridge by which the mounting plate is secured to the main body chassis; FIG. 29b is a top perspective view of the bladder and mesh assembly, the cartridge mounting plate, and magnets of the cartridge seen in FIG. 29a; FIG. 29c is a back perspective view of the bladder and the mesh assembly, the cartridge mounting plate, and magnets of the cartridge of FIG. 29a; FIG. 29d is a perspective elevational view of the bladder and the mesh assembly, the cartridge mounting plate, and magnets of the cartridge of FIG. 29a; FIG. 29e is another back perspective view of the bladder and the mesh assembly, the cartridge mounting plate, and magnets of the cartridge of FIG. 29a; FIG. 29f is a back elevational view of the bladder and the mesh assembly, the cartridge mounting plate, and magnets of the cartridge of FIG. 29a; FIG. 30a is a front perspective view of the bladder and the mesh assembly of FIG. 29a without the cartridge mounting plate and magnets; FIG. 30b is a bottom perspective view of the bladder and the mesh assembly of FIG. 30a; FIG. 30c is a back perspective view of the bladder and the mesh assembly of FIG. 30a; FIG. 30d is a back perspective view of the mesh assembly of FIG. 30a without the bladder; FIG. 30e is a back perspective view of the bladder of FIG. 30a without the mesh assembly; FIG. 30f is a bottom plan view of the bladder of FIG. 30e; FIG. 30g is a side elevational view of the bladder of FIG. 30e; FIG. 30h is a bottom perspective view of the bladder of FIG. 30e; and FIG. 30i is a top plan view of the bladder of FIG. 30a.

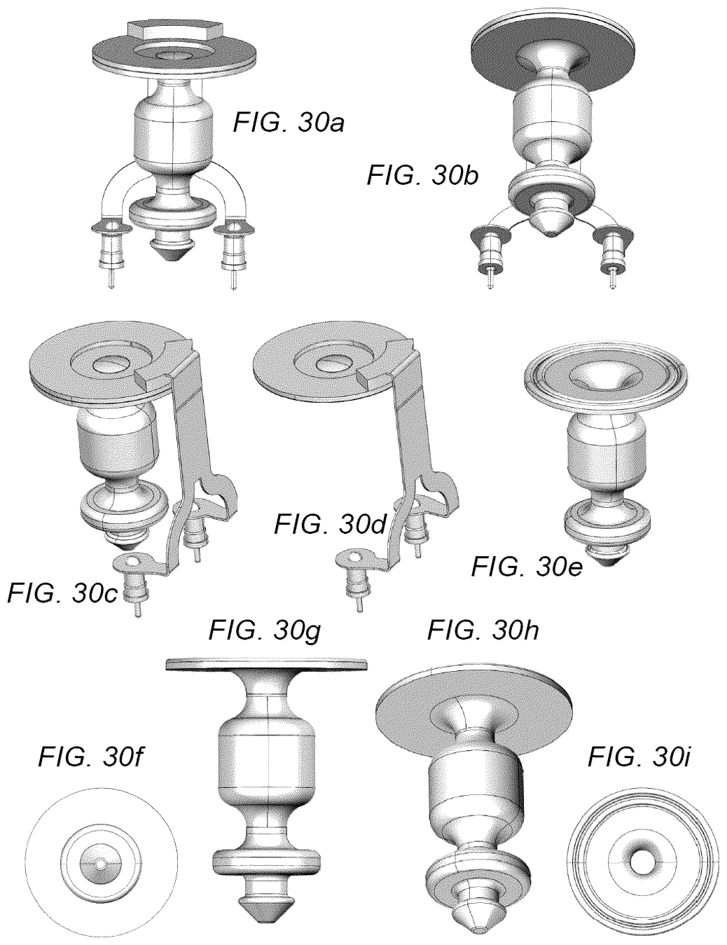

Other alternatives to the cartridges and bladders disclosed above are contemplated within the scope of the present invention and, indeed, are contemplated as forming part of other preferred embodiments of electronic devices of the invention. For example, FIG. 31a is a bottom perspective view of an alternative bladder 62 secured to the cartridge mounting plate 64 of FIG. 29a; and FIG. 31b is an exploded view of the alternative bladder 62 and mounting plate 64 of FIG. 31a.

FIG. 31c is yet another alternative bladder 62 secured to the cartridge mounting plate 64 of FIG. 29a, which view is a shaded line drawing; and FIG. 31d is a solid view of the view of FIG. 31c.

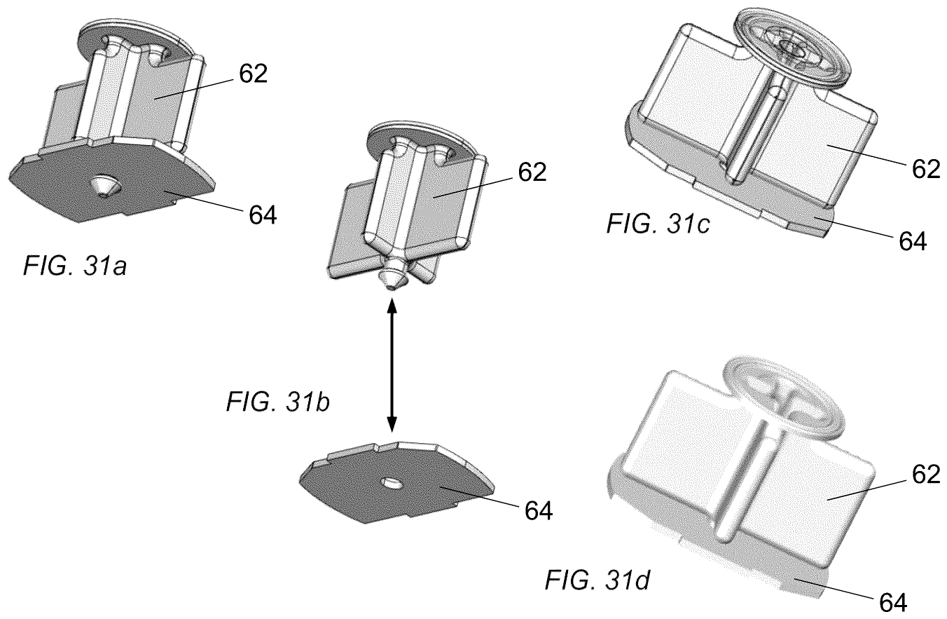

Additionally, FIG. 32a is a top perspective view of another alternative bladder 66 for use with the cartridge mounting plate of FIG. 29a; FIG. 32b is a bottom perspective view of the bladder of FIG. 32a; FIG. 32c is a top perspective view of another alternative bladder 68 for use with the cartridge mounting plate of FIG. 29a; FIG. 32d is a bottom perspective view of the bladder 68 of FIG. 32c; FIG. 32e is a top perspective view of another alternative bladder 70 for use with the cartridge mounting plate of FIG. 29a; and FIG. 32f is a bottom perspective view of the bladder 70 of FIG. 32e.

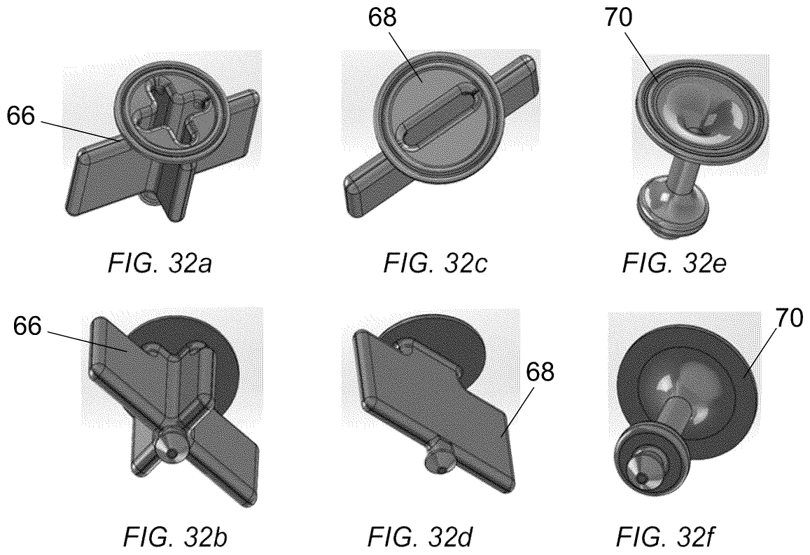

With particular regard to bladder shapes and geometries, including corrugated bladders, FIG. 33a is a top plan view of another alternative bladder 72 for use with the cartridge mounting plate of FIG. 29a; FIG. 33b is a bottom perspective view of the bladder 72 of FIG. 33a; FIG. 33c is a top plan view of another alternative bladder 74 for use with the cartridge mounting plate of FIG. 29a; FIG. 33d is a bottom perspective view of the bladder 74 of FIG. 33c; FIG. 33e is a top plan view of another alternative bladder 76 for use with the cartridge mounting plate of FIG. 29a; FIG. 33f is an elevational side view of the bladder 76 of FIG. 33e; FIG. 33g is a top plan view of another alternative bladder 78 for use with the cartridge mounting plate of FIG. 29a; and FIG. 33h is a bottom perspective view of the bladder 78 of FIG. 33g.

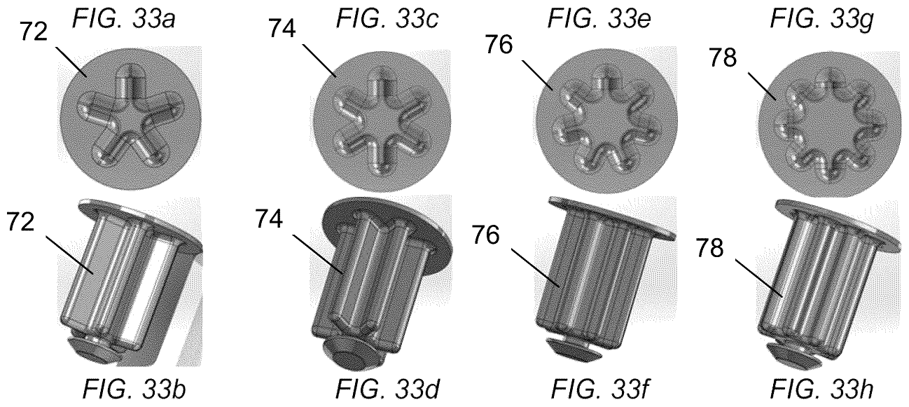

An example of a vaporizer 80 utilizing the bladder of FIG. 31a is seen in FIG. 34a, which is a wire frame illustration of the vaporizer illustrating in solid view use of the bladder of FIG. 31a. Furthermore, FIG. 34b is a transparent, top perspective view of a cartridge body 82 including mesh assembly illustrating in solid view use of the bladder of FIG. 31a; and FIG. 34c is a bottom perspective view of the cartridge body 82 of FIG. 34b.

FIG. 34d is a bottom perspective view of a cartridge 84 illustrating in solid view use of the bladder of FIG. 31c. FIG. 34e is a top perspective view of the cartridge of FIG. 34d.

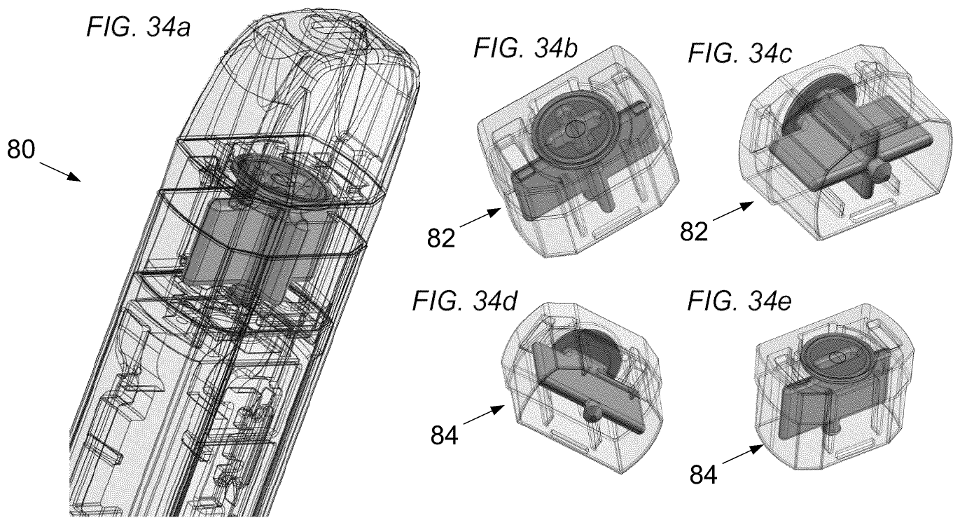

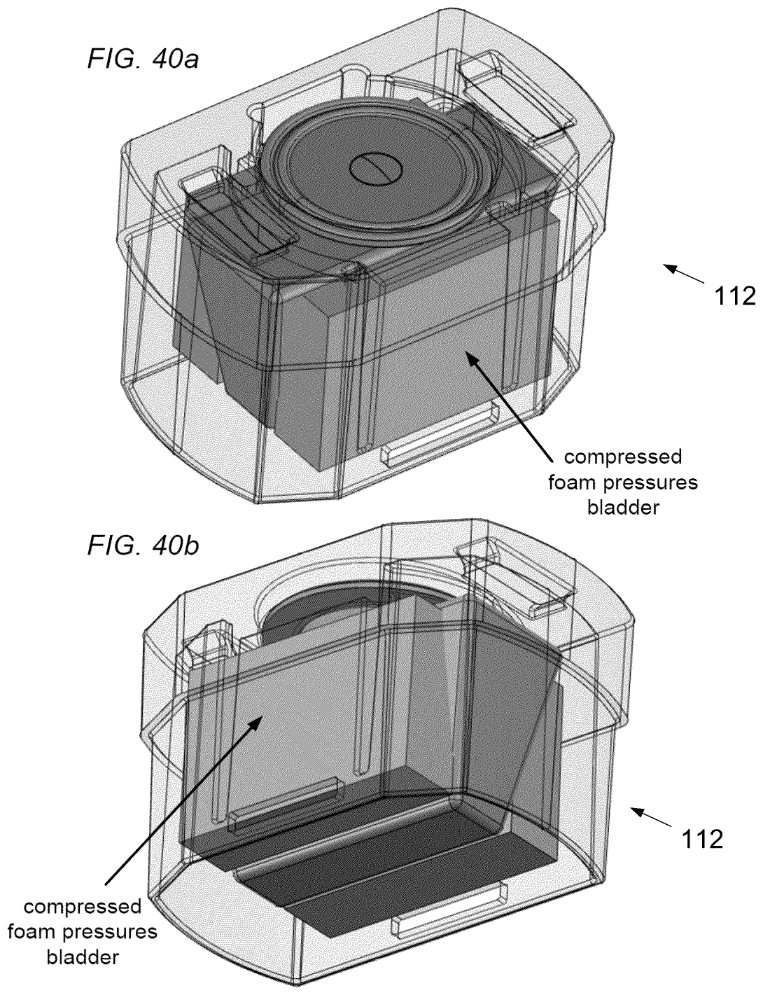

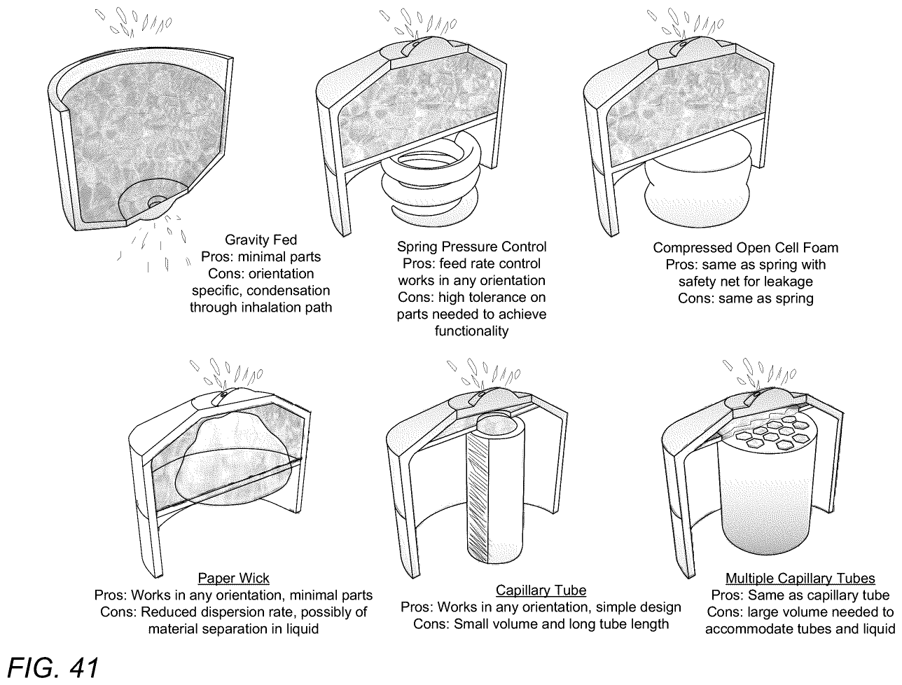

Different various methodologies for supplying liquid to the mesh assembly at a generally uniform pressure and so as to keep the liquid in continuous contact with the mesh material are disclosed in the alternative embodiments of cartridges seen in FIGS. 35a through 40b. FIG. 41 additionally sets forth other potential means for causing the liquid to contact the mesh material, which are shown in contrast to gravity fed systems. FIG. 42 additionally illustrates four additional low pressure bladder concepts that are contemplated for use in some preferred embodiments of the invention.

For example, FIG. 35a is a perspective view of an alternative cartridge 90 for use with the vaporizer of FIG. 23 illustrating in solid view a piezoelectric material, mesh material, bladder, and a mechanism for driving fluid from the bladder to the mesh material for aerosolizing; FIG. 35b is a perspective view of the other side of the cartridge of FIG. 35a; and FIG. 35c is a side elevational view of the cartridge of FIG. 35a.

FIG. 35d is a perspective view of an alternative cartridge 92 for use with the vaporizer of FIG. 23 illustrating in solid view a piezoelectric material, mesh material, bladder, and a mechanism for driving fluid from the bladder to the mesh material for aerosolizing. FIG. 35e is a view of the cartridge of FIG. 35d without the mechanism of FIG. 35d; and FIG. 35f is another view of the cartridge of FIG. 35e from a side opposite to the side of the view of FIG. 35e.

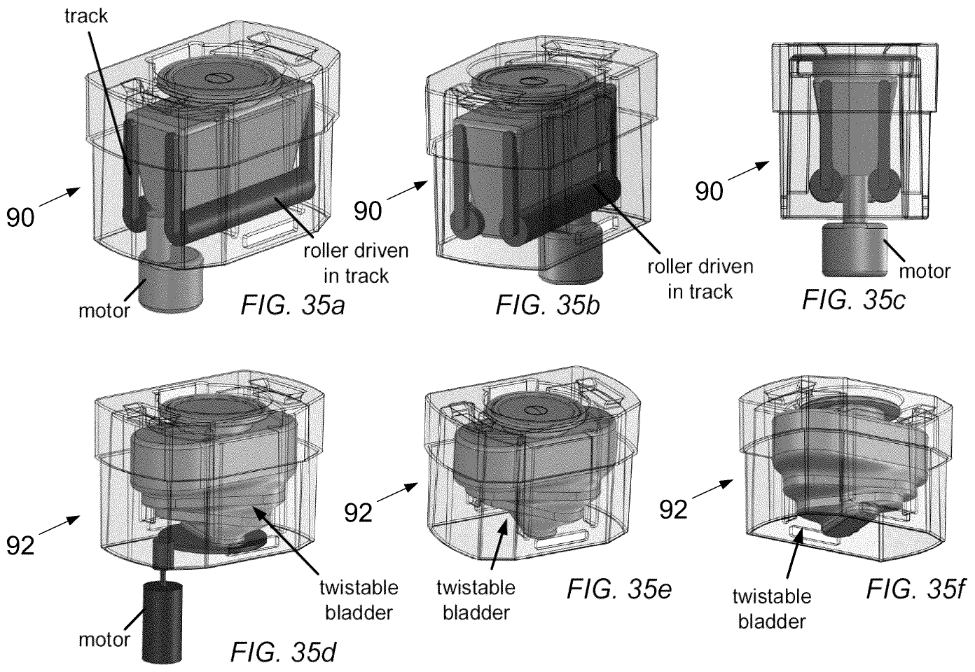

FIG. 36a is a top perspective view of an alternative cartridge 94 for use with the vaporizer of FIG. 23 illustrating in solid view a piezoelectric material, mesh material, bladder, and a mechanism for driving fluid from the bladder to the mesh material for aerosolizing. FIG. 36b is a bottom perspective view of the cartridge of FIG. 36a.

FIG. 36c is a top perspective view of an alternative cartridge 96 for use with the vaporizer of FIG. 23 illustrating in solid view a piezoelectric material, mesh material, bladder, and a mechanism for driving fluid from the bladder to the mesh material for aerosolizing.

FIG. 36d is a bottom perspective view of yet another alternative cartridge 98 for use with the vaporizer of FIG. 23 illustrating in solid view a piezoelectric material, mesh material, bladder, and a mechanism for driving fluid from the bladder to the mesh material for aerosolizing. FIG. 36e is a top perspective view of the cartridge of FIG. 36d without the mechanism of FIG. 36d.

FIG. 36f is a top perspective view of yet another alternative cartridge 100 for use with the vaporizer of FIG. 23 illustrating in solid view a piezoelectric material, mesh material, bladder, and a mechanism for driving fluid from the bladder to the mesh material for aerosolizing.

FIG. 37a is an elevational view of an alternative cartridge 102 for use with the vaporizer of FIG. 23 illustrating in solid view a bladder thereof. FIG. 37b is a top perspective view of the cartridge of FIG. 37a; and FIG. 37c is a bottom perspective view of the cartridge of FIG. 37a. The bladder, which folds or collapses in an accordion-like fashion, preferably comprises folds lines and is made of silicone.

FIG. 37d is a bottom perspective view of yet another alternative cartridge 104 for use with the vaporizer of FIG. 23 illustrating in solid view a bladder thereof which is similar to the bladder of FIG. 37a, but which includes a radial arm for side filling of liquid through injection at an injection site that is on a side of the cartridge rather than at the bottom of the cartridge. FIG. 37e is a top perspective view of the cartridge of FIG. 37d.

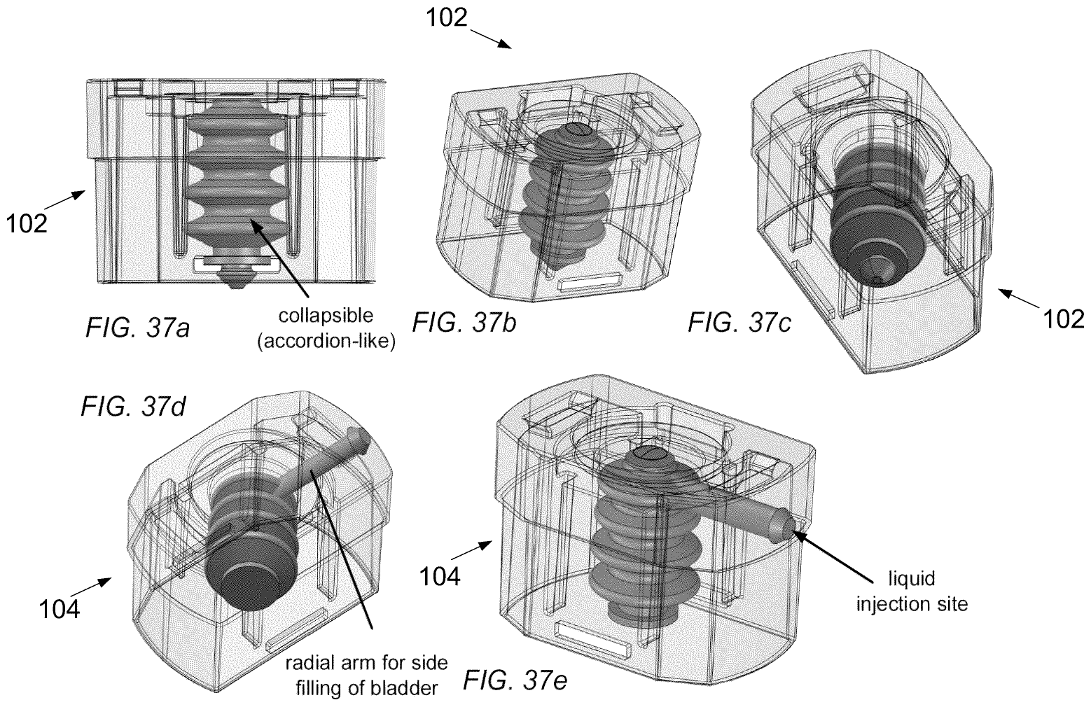

FIG. 38a is top perspective view of an alternative cartridge 106 for use with the vaporizer of FIG. 23 illustrating in solid view a piezoelectric material, mesh material, and bladder thereof; and FIG. 38b is a bottom perspective view of the cartridge of FIG. 38a. The bladder of these figures has a large volume is shown open or exposed. A mechanism for pressing against the bladder for expelling or driving the liquid therein into constant contact with the mesh material of the piezo mesh disk is preferably utilized, such mechanism being any of those disclosed herein.

FIG. 38c is top perspective view of yet another alternative cartridge 108 for use with the vaporizer of FIG. 23 illustrating in solid view a piezoelectric material, mesh material, and bladder thereof. FIG. 38d is another top perspective view of the cartridge of FIG. 38c; and FIG. 38e is a bottom perspective view of the cartridge of FIG. 38c. The bladder of this cartridge is the same as that of FIGS. 38a-38b with the exception that the bladder is contained within a chamber the conforms to the filled shape of the bladder. The chamber is open at the bottom, as seen in FIG. 38e. Use of a chamber facilitates use of a fluid, such as a gas or a secondary liquid, to be used for pressuring and collapsing the bladder.

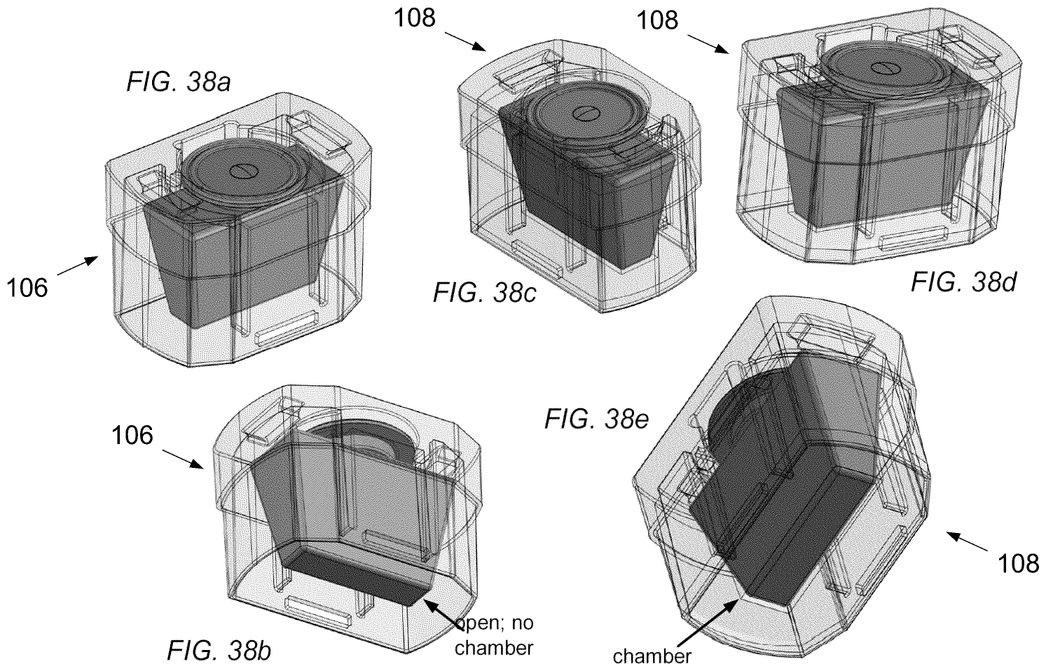

FIG. 39a is a top perspective view of an alternative cartridge 110 for use with the vaporizer of FIG. 23 illustrating in solid view piezoelectric materials, mesh material, and bladder thereof; and FIG. 39b is a bottom perspective view of the cartridge of FIG. 39a. The cartridge in these figures comprises a piezoelectric array located around the bladder. Each member of the array preferable is actuated in a sequence that drives the liquid toward the mouth of the bladder into contact with the mesh material of the piezo mesh disk. For example, the sequence of actuation can constrict the bladder beginning at the distal end with the constriction working its way toward the mouth, similar to intestinal movement.

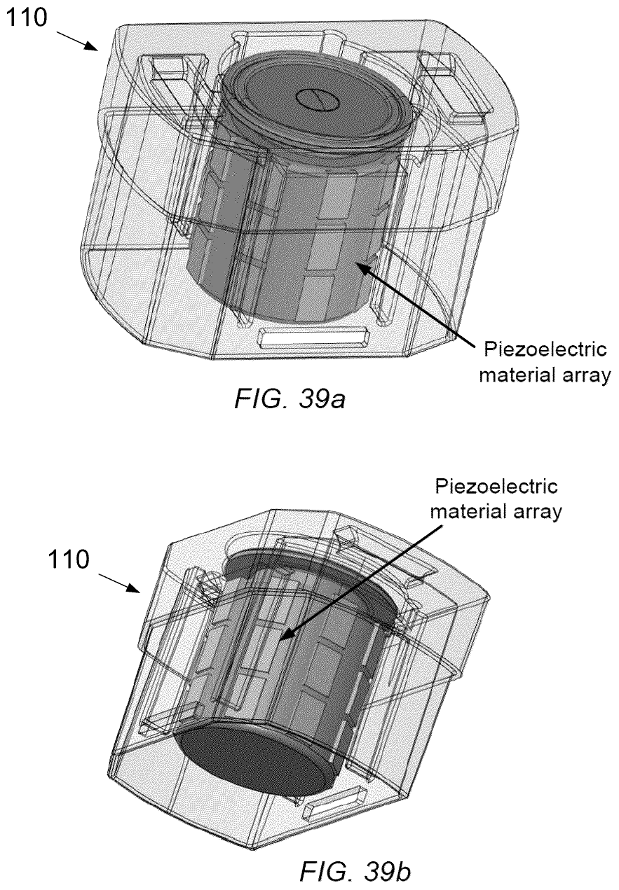

FIG. 40a is a top perspective view of an alternative cartridge 112 for use with the vaporizer of FIG. 23 illustrating in solid view a piezoelectric material, mesh material, bladder, and foam inserts or blocks.

FIG. 40b is a bottom perspective view of the cartridge of FIG. 40a. This cartridge comprises foam that is compressed when the bladder is filled. The compression of the foam preferable will pressure the bladder and drive liquid into constant contact with the mesh material of the piezo mesh disk. The foam may be an open cell foam.

Other contemplated ways of pumping, pushing, or otherwise forcing the liquid into contact with the vibrating mesh include using a solenoid pump, a capillary tube or plurality of capillary tubes, and a vacuum pump. Gravity may also be used when the electronic device is not intended to be orientation-agnostic in use, but such use is not preferred.

In each instance regardless of the manner in which the liquid is pushed from the cartridge into contact with the vibrating mesh, the liquid preferably is supplied to the vibrating mesh at a generally constant pressure whereby a generally uniform aerosol is produced. This is preferably done regardless of the orientation of the electronic device. The electronic device also preferably comprises a reservoir for the liquid. In some embodiments, the reservoir is an anti-pyrolysis vape reservoir with no smoldering and no combustion. In some embodiments, the liquid of the device features a thermostable liquid carrier.

Circuitry shown in the form of a printed circuit board or “PCB” in FIG. 28d, for example, preferably is included in each electronic device for controlling actuation of the vibrating mesh. A printed circuit board may comprise an application specific integrated circuit. The actuation and resulting vibrations/oscillations preferably are consistent for consistently generating the aerosol, with minimal variations or fluctuations in frequency and amplitude. The circuitry also preferably controls actuation of the mechanism—when an active mechanism—for pushing the liquid into contact with the vibrating mesh at a generally constant pressure. A microcontroller also may be included.

Additionally, with regard to more aspects and features of the present invention, the mesh material preferably has opening diameters of 1-2 microns, or 1,000-2,000 nanometers; when actuated, the flow rate of the liquid from the bladder to the vibrating mesh material is preferably about 0.25 milliliters per minute; preferred overall dimensions of a vaporizer are about 16 millimeters by 25 millimeters by 110 millimeters; and a vibrating mesh preferably remains in direct contact with the liquid for consistent production of the aerosol.

Furthermore, the bladder preferably physically contacts and forms a seal with the piezo mesh desk so that liquid from the bladder does not leak from the bladder. Specifically, a top flange of the bladder preferably services as a gasket or glad seal to the underside of the piezo mesh disk. The bottom end of the bladder preferably comprises the fill site and also serves to anchor and mechanically hold bladder in its position within the cartridge.

Additionally, the bladder preferably contains the volume of liquid behind the mesh at a relatively low pressure regardless of orientation as the volume is depleted with use. The seal is believed to fail at pressures above about 0.3 psi and, therefore, the liquid preferably is driven from the bladder into contact with the mesh material at less than 0.3 psi. The pressure at which the liquid is supplied to the mesh material therefore has been found to be very low.

When the liquid is an aqueous mixture (e.g., water or saline), the liquid preferably contacts the mesh material at a pressure less than about 0.3 psi. It is believed that higher pressure will lead to leaking and failure of the mesh material to produce a desired aerosol consistent with a vapor cloud. Higher pressure indeed may likely lead to the fluid flowing through perforations of the mesh material and flooding or wetting the other side of the mesh material causing the mesh material to not function correctly in producing the desired aerosol until dry. Higher pressure also may result in stretching and deformation of the wall so the bladder, resulting in mechanical failure.

The bladder also preferably acts as a capillary pump in addition to serving as a reservoir for the liquid.

Many preferred electronic devices of the invention are orientation agnostic, meaning that the desired aerosol is produced regardless of the direction an electronic device is held when used relative to the forces of gravity.

Disposable cartridges in accordance with preferred embodiments of the invention each comprises a mesh assembly including aperture plate and electronic contacts, a bladder, and a mouthpiece.

In accordance with a feature of the invention, the bladder has a hardness of about 40 durometer.

While alternatives such as pumps, drives, pistons, and wicking solutions are disclosed for causing the liquid in the bladder to be in contact with the mesh material, the bladder is believed to be the simplest mechanism and therefore is preferred. Furthermore, the bladder preferably is formed from a self-healing silicone material and can be filled with a syringe without injecting air into the bladder. This process preferably is automated and occurs during assembly of cartridges and/or vaporizers.

In some preferred embodiments, the bladder comprises corrugated walls and is formed from a flexible material and has flexibility such that the bladder changes volume without initially stretching of the material from which it is formed. Moreover, the volume of the bladder preferably is extremely low of nothing when the bladder is in a natural, fully relaxed (or collapsed) state. When fully collapsed, the bladder preferably provides some capillary benefit to extract the last amount of liquid from the bladder. To this end, it is believed that the vibrating mesh is able to create a light vacuum which is instrumental in fully evacuating the bladder of the liquid contained therein.

In some preferred embodiments of the invention, the electronic device comprises a vibrating mesh nebulizer coupled with a capillary-effect/vacuum pump system (corrugated silicone bladder), that acts as an orientation agnostic “liquid drive”, whereby the vaporizer is able to be held in any direction and still function properly.

In at lease some preferred embodiments, the corrugated bladder acts both as the capillary/vacuum pump as well as the liquid reservoir, ensuring that the liquid is in constant contact with the vibrating mesh, without disturbing the oscillations of the mesh material when the piezoelectric material is actuated.

Bladders disclosed also are believed to provide a range of interior surface areas relative to volume and pressure for desired supply of the liquid to the mesh material and proper operation of the vaporizer. Indeed, other shapes and geometries may not enable the capillary action of the bladder, the orientation-agnostic character of the operation of the vaporizer, and the proper oscillation of the mesh material (i.e., too much pressure and leaking of the fluid can mute oscillations of the mesh material, inhibiting aerosolizing of the liquid in a vaping form). Flexibility of design also allows corrugated walls and flexibility of the material the bladder allows changes in volume without initially stretching the material, which is preferred. Thus, in at least some preferred embodiments, the volume of a natural state or relaxed state of the bladder is basically zero. When all fully collapses some capillary benefit should be obtained in order to extract last amount of liquid from the bladder, especially when combined with the light vacuum provided by the vibrating mesh.

Additionally, while the top flange of the bladder serves as a gasket or glad seal to the underside of the mesh, the bottom distal end or “post” of the bladder that preferably serves the fill sight also mechanically holds the bladder in position when secured to the mounting plate of the cartridge.

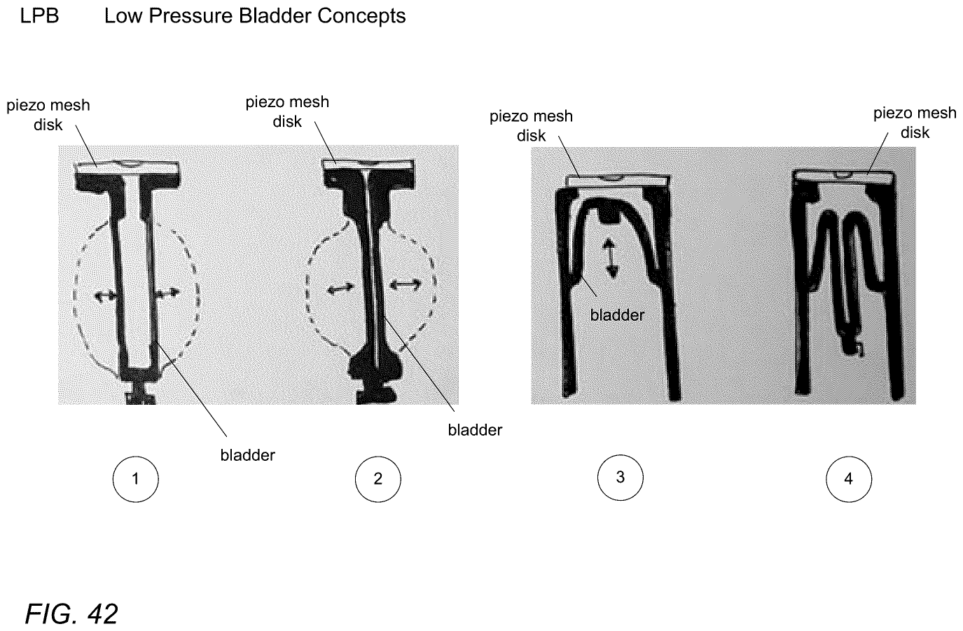

With regard to additional aspects, features and embodiments of the invention, and with reference to FIGS. 43-45, a preferred active ingredient delivery system for inhalation is contemplated to be capable of accommodating and delivering a range of different types of active ingredients to the body through the pulmonary system. Active ingredients capable of delivery using one or more delivery systems described herein include, but are not limited to, pharmaceutical compounds, tetrahydrocannabinol (THC), cannabidiol (CBD), and nicotine. The following description of embodiments sets forth one or more active ingredient delivery systems largely within the context of delivering THC and/or CBD, but it should be understood that active ingredient delivery systems described herein are also usable for delivery of nicotine, pharmaceuticals, micronutrients, and other types of active ingredients by inhalation and are not limited to delivery of THC/CBD.

THC and CBD are two of several different cannabinoids found in plants of the Cannabis genus. Using extraction techniques, THC and CBD can be isolated from the plant matrix for medicinal and/or recreational use. THC and CBD interact with different receptors in the human brain and, thus, cause a different treatment or effect in the user. For purposes of the below discussion, THC and CBD may be referenced together as “THC/CBD.” It should be understood that, as used herein, “THC/CBD” refers to a cannabinoid-based active ingredient that includes both THC and CBD, THC without CBD, or CBD without THC.

THC and CBD are hydrophobic molecules that do not readily mix with aqueous solutions like water. To facilitate delivery to the human body, THC/CBD molecules are encapsulated into nanoparticles comprising oil droplets of the THC/CBD active ingredient surrounded by one or more encapsulation agents, such as surfactants or emulsifiers, which shield the oil droplets from the surrounding aqueous environment. The shielded oil droplets can then mix into aqueous solutions. One example of such a mixture is a nanoemulsion, where the oil phase includes the hydrophobic THC/CBD molecules shielded by one or more surfactants from the surrounding aqueous phase.

FIG. 43 is a schematic diagram of an active ingredient pulmonary delivery nanoparticle in the form of a micelle 810 in accordance with one or more aspects of the invention. In FIG. 43, the hydrophobic droplet 812 comprised of oil containing THC/CBD molecules is surrounded by a monolayer 814 of one or more encapsulation agents, which forms an aggregate. In at least some embodiments, the monolayer 814 is a lipid-based monolayer. Molecules forming the monolayer 814 include hydrophilic heads 816 that are in contact with the surrounding aqueous solution 840 and hydrophobic tails 818 that extend toward the micelle center. The hydrophilic heads 816 form the boundary of the monolayer 814 that facilitates isolation of the hydrophobic component, including the hydrophobic active ingredient 860, to permit mixing of the micelle 810 into the aqueous solution 840. As shown in FIG. 43, the micelle 810 is largely spherical in shape, although non-spherical shapes are also possible. As shown in FIG. 43, the micelle 810 and the aqueous solution 840 are contained within a cartridge 800.

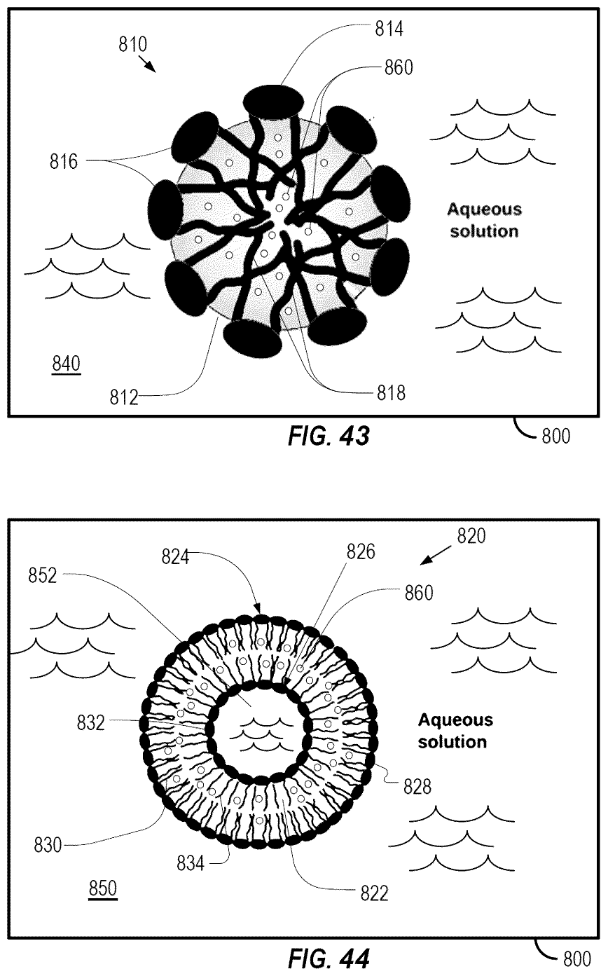

FIG. 44 is a schematic diagram of an active ingredient pulmonary delivery nanoparticle in the form of a liposome 820 carrying an active ingredient 860 within a bilayer in accordance with one or more aspects of the invention. In FIG. 44, the oil component resides in a hydrophobic area 822 of the liposome 820 between a bilayer of one or more encapsulation agents. In at least some embodiments, the bilayer is a lipid-based bilayer. Molecules that form the outer layer 824 of the bilayer include hydrophilic heads 828 that are in contact with the surrounding aqueous solution 850 and hydrophobic tails 830 that extend into the hydrophobic area 822 between the layers 822,824. Lipid molecules that form the inner layer 826 of the bilayer include hydrophilic heads 832 that are in contact with the aqueous solution 852 at the center of the liposome 820 and hydrophobic tails 834 that extend into the hydrophobic area 822 of the bilayer. The hydrophilic heads 828,832 form the boundaries of the bilayer that facilitate isolation of the hydrophobic area, which includes the hydrophobic active ingredient 860. With the hydrophobic area 822 isolated, the liposome 820 can be mixed into the surrounding aqueous solution 850. As indicated in FIG. 44, the liposome 820 is largely spherical in shape, although non-spherical shapes are also possible. As shown in FIG. 44, the liposome 820 and the surrounding aqueous solution 850 are contained within a cartridge 800.

Liquid mixtures that include active ingredient delivery nanoparticles in accordance with FIG. 43 or 44 include an active ingredient, an encapsulation agent, and an aqueous solution. As described herein, one contemplated active ingredient includes THC/CBD molecules, although a wide range of other active ingredients are contemplated to be deliverable to the human pulmonary system in accordance with the invention, including, but not limited to, pharmaceutical compounds, micronutrients, and nicotine. Encapsulation agents to encapsulate hydrophobic active ingredient molecules are compounds with a hydrophobic region and a hydrophilic region. It is contemplated that encapsulation agents include, but are not limited to, lipids, polymers, and surfactants. Encapsulation agents can be used singly or in combination with each other. The aqueous solution is a medium that can be selected and formulated to achieve an osmotic balance with respect to human physiology. In at least some embodiments, the aqueous solution is a 0.9% saline solution, which is understood to provide a preferred osmotic balance with human physiology of the lungs. Furthermore, a 0.9% saline solution as the aqueous medium facilitates a safer user experience, particularly when the liquid mixture is aerosolized.

With respect to polymers as encapsulation agents, it is contemplated that polymers include, but are not limited to, poly(lactic-co-glycolic) acid (PLGA), polylactic acid (PLA), polyglycolic acid (PGA), polycaprolactone (PCL), and polyhydroxybutyrate (PHB).

With respect to surfactants as encapsulation agents, it is contemplated that surfactants include, but are not limited to: high purity polyoxyethylene sorbitan monooleate (also known by its trade name, SUPER REFINED® Polysorbate 80); polyoxyethylene sorbitan monooleate; (also known by its trade name, TWEEN® Polysorbate 80); polyoxyethylene sorbitan monostearate (also known by its trade name TWEEN® Polysorbate 60); polyoxyethylene sorbitan monopalmitate (also known by its trade name TWEEN® Polysorbate 40); polyoxyethylene sorbitan monolaurate (also known by its trade name TWEEN® Polysorbate 20); lecithin; dipalmitoylphosphatidylcholine (DPPC); 1,2-distearoyl-sn-glycero-3-phosphocholine (DSPC); sorbitan monostearate (also known by its trade name SPAN 60); and sorbitan monopalmitate (also known by its trade name SPAN 40). When using one or more surfactants as an encapsulating agent, a ratio of surfactant combinations is determined by hydrophilic-lipophilic balance (HLB) values inherent to each surfactant. The combination of surfactants yields a weighted average HLB value that can be used to match the target application in order to enhance or optimize mixing of nanoparticles containing the active ingredient into the aqueous solution. For example, an HLB value measuring from approximately 8 to approximately 16 is satisfactory for oil-in-water emulsions.

In at least some embodiments, the encapsulating agent includes a high purity or high-grade surfactant, which is understood to enhance the shelf-life of the resulting mixture as well as to improve the efficacy and safety of the resulting mixture. One such high purity surfactant that can be used in the formulation is high purity polyoxyethylene sorbitan monooleate, which is also known by its trade name, SUPER REFINED® Polysorbate 80. SUPER REFINED® Polysorbate 80 is manufactured and sold by Croda International Plc of the United Kingdom.

A ratio of the surfactant relative to the active ingredient affects the size of the resulting nanoparticles (e.g., micelles and/or liposomes that contain the active ingredient). In various embodiments, it is contemplated that the surfactant-to-active-ingredient ratio can range from approximately 0.1:1 to approximately 10:1. Size of the resulting nanoparticles that contain the active ingredient affects a variety of characteristics of the final product, including pulmonary deposition of the active ingredient, absorption of the active ingredient, and the product shelf-life.

In at least some embodiments, a process for producing a liquid mixture that includes active-ingredient nanocarriers in accordance with FIGS. 43-45 is accomplished using a microfluidics approach. Microfluidics involves utilizing a network of channels having very small dimensions to process the liquid mixture in order to achieve homogeneous mixture with consistently-sized nanoparticles. In one such embodiment, a microfluidizer is utilized to achieve the desired nanoparticle dispersal and uniform mixture with consistently-sized nanoparticles. During a processing step using a microfluidizer, it is contemplated that a temperature of the liquid mixture does not exceed a temperature threshold of 65° C. By not exceeding a predetermined temperature threshold, the risk of generating harmful HPHCs in the mixture via heat is reduced, thereby enhancing consumer safety. Additionally, processing the liquid mixture using a microfluidizer facilitates processing without the use of chemical solvents, which further reduces the risk of generating harmful HPHCs in the final liquid mixture. Still further, use of a microfluidics approach helps to maintain sterility in the materials used to produce the final liquid mixture, which also enhances consumer safety.

Using a microfluidics approach, the processed liquid includes nanoparticles of a uniformly small size and a low polydispersity index (PDI) value. In at least some embodiments, it is contemplated that THC/CBD nanoparticles in the final liquid mixture have an average diameter less than 1,000 nanometers or, alternatively, have a dimension that is no larger than 1,000 nanometers. It is believed that nanoparticles of this scale provide enhanced pulmonary deposition of the active ingredient into the alveolar lung region, which facilitates increased pulmonary absorption. Furthermore, nanoparticles of this scale enhance the stability of the final liquid mixture, which increases its shelf-life. Additionally, in at least some embodiments, it is contemplated that the final liquid mixture has a PDI value measuring less than 0.3. The PDI value provides a measurement of the broadness of size distribution. A low PDI value is indicative of a high level of particle size uniformity in a mixture. In accordance with contemplated embodiments of the invention, the PDI value is 0.3 or less, which is believed to indicate a liquid mixture with increased stability and enhanced shelf-life. A PDI measurement scale assigns a value of 0.0 to a population of particles where the particles have a perfectly uniform size and a value of 1.0 to a highly polydisperse population of particles with multiple size populations.

In at least some embodiments, it is contemplated that the pH of the final liquid mixture can be adjusted to accommodate a specific objective. For example, in some embodiments, a pH value of the final liquid mixture that is greater than approximately 3 and less than approximately 10 can improve the inhalation experience for the user by reducing a cough reaction. In preferred embodiments, a pH value of the final liquid mixture that is greater than approximately 5.5 and less than approximately 8, more preferably, is about 6.5, so as to match the pH of the human respiratory tract, improve consumer safety, enhance pulmonary absorption of the active ingredient, and enhance or optimize shelf-life of the liquid.

The final liquid mixture includes many THC/CBD-encapsulated nanoparticles that are uniformly suspended in an aqueous solution for downstream aerosolization by an aerosolizing device for inhalation. Such devices may include, for example, vaporizers and nebulizers.

In at least some embodiments, the encapsulated molecules are chemically bonded to other molecules in a conjugated system. Establishing a conjugated system with chemical bonds between the active ingredient molecules and other molecules facilitates more efficient encapsulation of the active ingredients via the techniques described herein. In some contemplated embodiments, then THC/CBD molecules are chemically bonded with molecules of stearic acid and/or oleic acid. Establishing a conjugated system, as described herein, is understood to enhance or optimize encapsulation of THC/CBD molecules as well as other drugs or pharmaceutical compounds.

It is contemplated that formulations and methods as described herein can be applied to hydrophobic drugs or compounds other than THC/CBD. It is further contemplated that formulations and methods as described herein can be applied to hydrophilic drugs or compounds with modifications. One such modification includes encapsulating the hydrophilic drug or compound into a hydrophilic core of a liposomal nanoparticle. Another such modification includes conjugation of the hydrophilic drug or compound to a hydrophobic molecule (such as by chemical bonding) in order to achieve an overall hydrophobic compound capable of being encapsulated in the manner as set forth in FIGS. 43 and 44.

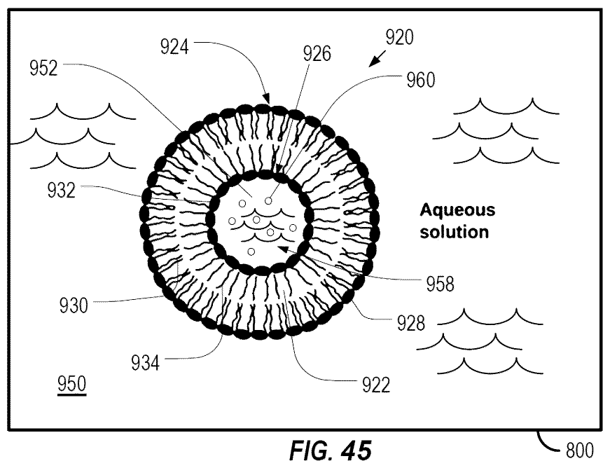

Regarding encapsulation of a hydrophilic drug or compound into a hydrophilic core of a liposomal nanoparticle, reference is made to FIG. 45, which is a schematic diagram of an active ingredient pulmonary delivery nanoparticle in the form of a liposome 920 carrying a hydrophilic active ingredient 960 in a hydrophilic core 958 in accordance with one or more aspects of the invention. In FIG. 45, the hydrophobic component resides in a hydrophobic area 922 of the liposome 920 between a bilayer of one or more encapsulation agents. In at least some embodiments, the bilayer is a lipid-based bilayer. Molecules that form the outer layer 924 of the bilayer include hydrophilic heads 928 that are in contact with the surrounding aqueous solution 950 and hydrophobic tails 930 that extend into the hydrophobic area 922 of the bilayer. Lipid molecules that form the inner layer 926 of the bilayer include hydrophilic heads 932 that are in contact with the aqueous solution 952 at the core 958 of the liposome 920 and hydrophobic tails 934 that extend into the hydrophobic area 922 of the bilayer. The hydrophilic heads 928,932 form the barriers of the bilayer that facilitate isolation of the hydrophobic area 922. The hydrophilic active ingredient 960 is contained within the hydrophilic core 958. With the hydrophobic area 922 isolated, the liposome 920 can be mixed into the surrounding aqueous solution 950. As indicated in FIG. 45, the liposome 920 is largely spherical in shape, although non-spherical shapes are also possible. Also, the liposome 920 and the surrounding aqueous solution 950 are contained within a cartridge 800.

In at least some embodiments, it is further contemplated that the aqueous solution of the product can be buffered to mitigate pH over time. In this respect, it is contemplated that a saline solution can be converted to a phosphate buffer saline solution. Buffering the solution with the addition of a buffering agent can enhance consistency of the product, increase the shelf-life, and enhance the consumer experience when the product is aerosolized during use.

In at least some embodiments, it is further contemplated that additives can be included in the aqueous solution of the product. Contemplated additives include, but are not limited to antioxidants (such as ascorbic acid, sodium ascorbate, or others) and preservatives (such as antimicrobials). In some respects, additives can provide a safer consumer experience when the product is aerosolized during use. In other respects, additives can enhance the shelf-life of the product.

Additives can also be used to enhance or complement the user experience. For example, additives can be included to enhance or complement the smell/taste during inhalation of the aerosolized product. Additives to enhance or complement the smell/taste during inhalation include, but are not limited to, menthol and mint. Furthermore, additives can be included to enhance or complement the inhalation sensation during inhalation of the aerosolized product. An additive that enhances or complements the inhalation sensation might mimic a throat hit sensation commonly associated with nicotine inhalation or the sensation might trigger a feeling of smoothness for the consumer.

In at least some embodiments, it is further contemplated that a carrier or diluent solution is used in connection with the active ingredient to increase stability of the resulting product. Additionally, a carrier or diluent solution can enhance manufacturing process efficiency with respect to the ability to encapsulate the active ingredient when forming the nanoparticles. One contemplated carrier or diluent solution includes a medium-chain triglyceride (MCT) oil.

While many aspects and features relate to, and are described in, the context of THC/CBD delivery systems, the invention is not limited to use only in pulmonary delivery of THC/CBD, as will become apparent from the following summaries and detailed descriptions of aspects, features, and one or more embodiments of the invention.

Based on the foregoing description, it will be readily understood by those persons skilled in the art that the invention has broad utility and application. Electronic devices of the invention can be utilized to deliver liquids comprising supplements, drugs, or therapeutically effective amounts of pharmaceuticals using an aerosol having particles of a size that can easily be inhaled. The aerosol can be used, for example, by a patient within the bounds of an inhalation therapy, whereby the liquid containing a supplement, therapeutically effective pharmaceutical, or drug reaches the patient's respiratory tract upon inhalation. Desired compounds such as nicotine, flavoring, and supplements like B12, can be received by a person through inhalation without the toxic byproducts like formaldehyde—a recognized Group 1 Carcinogen for caner—that is currently being created during heating in conventional vapes. Electronic devices of the invention further can be used in the marijuana industries, but only where legal, for delivery of cannabinoids and CBD oils and the like. Moreover, many embodiments and adaptations of the invention other than those specifically described herein, as well as many variations, modifications, and equivalent arrangements, will be apparent from or reasonably suggested by the invention and the foregoing descriptions thereof, without departing from the substance or scope of the invention.

It further will be appreciated from the foregoing that at least some preferred embodiments of the invention represent a portable, orientation-agnostic vibrating mesh nebulizer. It further will be appreciated from the foregoing that at least some preferred embodiments emit an aerosol that is—sensorially speaking—equivalent to vapor, i.e., not a mist but instead that which is generated by traditional vapes, thereby providing an enjoyable consumer product for those who are accustomed to vaping.

Accordingly, while the invention has been described herein in detail in relation to one or more preferred embodiments, it is to be understood that this disclosure is only illustrative and exemplary of the invention and is made merely for the purpose of providing a full and enabling disclosure of the invention. The foregoing disclosure is not intended to be construed to limit the invention or otherwise exclude any such other embodiments, adaptations, variations, modifications or equivalent arrangements, the invention being limited only by the claims appended hereto and the equivalents thereof.

What is claimed is:

1. An electronic device for producing an aerosol for inhalation by a person, comprising:
(a) a mouthpiece;
(b) a bladder containing a liquid;
(c) a mesh assembly comprising a mesh material and a piezoelectric material, wherein the mesh material is configured to vibrate when the piezoelectric material is actuated whereby the aerosol is produced when the mesh material contacts the liquid of the liquid container such that the aerosol may be inhaled through the mouthpiece; and
(d) circuitry and a power supply for actuating the mesh assembly.

2. The electronic device of claim 1, wherein the bladder and mesh assembly are located in a removable cartridge of the electronic device.

3. The electronic device of claim 2, wherein the cartridge mounts onto an end of base housing of the electronic device, which base housing contains the circuitry and the power supply for actuating the mesh assembly.

4. The electronic device of claim 2, wherein the cartridge forms the mouthpiece of the electronic device.

5. The electronic device of claim 2, wherein electrical contacts of the mesh assembly interface with electrical contacts connecting to the circuitry and power supply when the cartridge is mounted onto the end of the base housing of the electronic device.

6. The electronic device of claim 1, wherein the bladder contains between about 1 ml and 3 ml of the liquid.

7. The electronic device of claim 1, wherein the mesh assembly comprises a piezo mesh disk.

8. The electronic device of claim 3, wherein the mesh assembly comprises an annular ring and wherein the mesh material is located within the area bounded by the annual ring.

9. The electronic device of claim 1, wherein the mesh material is flat.

10. The electronic device of claim 1, wherein the mesh material is dome-shaped.

11. The electronic device of claim 1, wherein the mesh material is constructed from a metal alloy.

12. The electronic device of claim 1, wherein the mesh material is produced by electroplating.

13. The electronic device of claim 1, wherein the mesh material is produced by laser cutting.

14. The electronic device of claim 1, wherein the mesh material is in the form of a mesh plate.

15. The electronic device of claim 1, further comprising a sensor that detects when a person inhales, and wherein the mesh assembly is actuated in response to the detection of inhalation by a person.

16. The electronic device of claim 1, wherein the cartridge is a single-use, disposable cartridge.

17. The electronic device of claim 1, wherein the cartridge further comprises a port through which the aerosolized liquid is inhaled.

18. The electronic device of claim 17, wherein the cartridge serves as a mouthpiece of the electronic device.

19. A liquid-filled cartridge for use with an electronic device for delivery of a substance into a body through respiration, comprising:
(a) a liquid container; and
(b) a liquid for aerosolizing and inhaling by a person using the electronic device, the liquid being contained within the liquid container and comprising a nanoemulsion, each of a plurality of nanoparticles of the nanoemulsion comprising an encapsulation of the substance to be delivered into the body through respiration.

20. The liquid-filled cartridge of claim 19, wherein the liquid is an oil-in-water nanoemulsion.

21. The liquid-filled cartridge of claim 19, wherein each nanoparticle is a micelle.

22. The liquid-filled cartridge of claim 19, wherein each nanoparticle is a liposome.

23. The liquid-filled cartridge of claim 19, wherein the substance is encapsulated by a polymer.

24. The liquid-filled cartridge of claim 19, wherein the substance is encapsulated by a surfactant.

25. The liquid-filled cartridge of claim 24, wherein the surfactant comprises high purity polyoxyethylene sorbitan monooleate.

26. The liquid-filled cartridge of claim 19, wherein the encapsulated substance comprises tetrahydrocannabinol.

27. The liquid-filled cartridge of claim 19, wherein the encapsulated substance comprises cannabidiol.

28. The liquid-filled cartridge of claim 19, wherein the encapsulated substance comprises tetrahydrocannabinol and cannabidiol.

29. The liquid-filled cartridge of claim 19, wherein the encapsulated substance comprises a pharmaceutical compound.

30. The liquid-filled cartridge of claim 19, wherein the encapsulated substance comprises nicotine.

31. The liquid-filled cartridge of claim 19, wherein the nanoparticles are dispersed within an aqueous solution.

32. The liquid-filled cartridge of claim 31, wherein the aqueous solution comprises a saline.

33. The liquid-filled cartridge of claim 32, wherein the aqueous solution comprises sodium chloride.

34. The liquid-filled cartridge of claim 33, wherein the nanoparticles are dispersed within an aqueous solution of 0.9% sodium chloride.

35. The liquid-filled cartridge of claim 19, wherein a pH of the liquid is between about 5.5 and about 8.

36. The liquid-filled cartridge of claim 19, wherein a pH of the liquid is between about 6.5.

37. The liquid-filled cartridge of claim 19, wherein a molecular ratio of the encapsulated substance to an encapsulating agent of the nanoparticle is between about 0.1:1 to about 10:1.

38. The liquid-filled cartridge of claim 19, wherein a polydispersity index measurement of the nanoemulsion is less than 0.3.

39. A method of manufacturing cartridges for use with an electronic device for delivery of a substance into a body through respiration, comprising filling a liquid container of the cartridge with a liquid for aerosolizing and inhaling by a person using the electronic device, the liquid comprising a plurality of nanoparticles in a nanoemulsion, each nanoparticle comprising an encapsulation of the substance to be delivered into the body through respiration.

40. The method of claim 39, further comprising a preliminary step of producing the nanoemulsion by processing the substance to be delivered together with the encapsulating agent using a microfluidizing machine.

41. The method of claim 40, further comprising operating the microfluidizing machine such that a temperature of the processing does not exceed 65° C. while producing the nanoemulsion.

42. The method of claim 40, further comprising the step of adjusting pH of the nanoemulsion so as to be between about 5.5 and 8.

43. The method of claim 40, further comprising the step of chemically bonding the substance to be encapsulated with another molecule prior to processing the substance with the encapsulating agent using the microfluidizing machine.

44. The method of claim 40, wherein a polydispersity index measurement of the nanoemulsion after processing using the microfluidizing machine is less than 0.3.

45. A method of manufacturing a liquid for aerosolizing and inhaling by a person using an electronic device for the delivery of a substance to the body of the person through respiration, the method comprising producing a liquid mixture comprising a plurality of nanoparticles in a nanoemulsion by processing the substance together with an encapsulating agent using a microfluidizing machine such that the plurality of nanoparticles of the nanoemulsion comprises the encapsulated substance.

46. The method of claim 45, further comprising operating the microfluidizing machine such that a temperature of the processing does not exceed 65° C. while producing the nanoemulsion.

47. The method of claim 45, further comprising the step of adjusting pH of the nanoemulsion so as to be between about 5.5 and 8.

48. The method of claim 45, further comprising the step of chemically bonding the substance to be encapsulated with another molecule prior to processing the substance with the encapsulating agent using the microfluidizing machine.

49. The method of claim 45, wherein a polydispersity index measurement of the nanoemulsion after processing using the microfluidizing machine is less than 0.3.
